SEQUENCE LISTING (22 sequences)

SEQ ID NO: 1 - DNA, 23 bp, Synthetic construct
  source 1..23: mol_type = other DNA
accattgttcaatatcgtccngg

SEQ ID NO: 2 - DNA, 120 bp, synthetic construct
  source 1..120: mol_type = other DNA
cttttcacccatctacagtcccccttgccgtcccaagcaatggatgatttgatgctgtccagtgacgatattgaacaatggttcactgaagacccaggtccagatgaagctcccagaatg

SEQ ID NO: 3 - DNA, 23 bp, synthetic construct
  source 1..23: mol_type = other DNA
aaaacctaccagggcagctacgg

SEQ ID NO: 4 - DNA, 124 bp, synthetic construct
  source 1..124: mol_type = other DNA
tgaccgtgcaagtcacagacttggctgtcccagaatgcaagaagcccagacggaaaccgtgggagccctggtaggttttctgggaagggacagaagatgacaggggccaggagggggctggtgc

SEQ ID NO: 5 - DNA, 24 bp, synthetic construct
  source 1..24: mol_type = other DNA
aaattcggtacatcctcgacggca

SEQ ID NO: 6 - DNA, 24 bp, synthetic construct
  source 1..24: mol_type = other DNA
agtgcctctttgctgctttcacac

SEQ ID NO: 7 - DNA, 20 bp, synthetic construct
  source 1..20: mol_type = other DNA
atgacttccaagctggccgt

SEQ ID NO: 8 - DNA, 20 bp, synthetic construct
  source 1..20: mol_type = other DNA
tccttggcaaaactgcacct

SEQ ID NO: 9 - DNA, 20 bp, synthetic construct
  source 1..20: mol_type = other DNA
cctacccgccacgatgatgt

SEQ ID NO: 10 - DNA, 20 bp, synthetic construct
  source 1..20: mol_type = other DNA
ctgcgggtgagtggtagtaa

SEQ ID NO: 11 - DNA, 20 bp, synthetic construct
  source 1..20: mol_type = other DNA
acctgaccgcagagtctttt

SEQ ID NO: 12 - DNA, 20 bp, synthetic construct
  source 1..20: mol_type = other DNA
gagtggtttggctggggtaa

SEQ ID NO: 13 - DNA, 21 bp, synthetic construct
  source 1..21: mol_type = other DNA
agacatgccaaagtgctgagt

SEQ ID NO: 14 - DNA, 20 bp, synthetic construct
  source 1..20: mol_type = other DNA
gaggctagcgctaagaagca

SEQ ID NO: 15 - DNA, 18 bp, synthetic construct
  source 1..18: mol_type = other DNA
tgggccatcagttgcaaa

SEQ ID NO: 16 - DNA, 24 bp, synthetic construct
  source 1..24: mol_type = other DNA
acttatgcaccctcatcttttcat

SEQ ID NO: 17 - DNA, 20 bp, synthetic construct
  source 1..20: mol_type = other DNA
agcaggctgtggctctgatt

SEQ ID NO: 18 - DNA, 23 bp, synthetic construct
  source 1..23: mol_type = other DNA
caaaatagccaccagcctcttct

SEQ ID NO: 19 - DNA, 20 bp, synthetic construct
  source 1..20: mol_type = other DNA
catccctggactgattgtca

SEQ ID NO: 20 - DNA, 20 bp, synthetic construct
  source 1..20: mol_type = other DNA
ggttggccaggtacatgttt

SEQ ID NO: 21 - DNA, 23 bp, synthetic construct
  source 1..23: mol_type = other DNA
aaaacctaccagggcagctacgg

SEQ ID NO: 22 - DNA, 124 bp, synthetic construct; identical to SEQ ID NO: 4
  source 1..124: mol_type = other DNA

(19) United States
(12) Patent Application Publication
(10) Pub. No.: US 2026/0357351 A1
(43) Pub. Date: Nov. 12, 2026

(54) COMPOSITIONS AND METHODS FOR IDENTIFICATION OF HYPOMORPHIC P53 VARIANTS

(71) Applicant: The Wistar Institute of Anatomy and Biology, Philadelphia, PA (US)

(72) Inventor: Maureen E. Murphy, Glenside, PA (US)

(21) Appl. No.: 19/138,994

(22) PCT Filed: Dec. 22, 2023
(86) PCT No.: PCT/US2023/085631

§ 371 Date: Jun. 13, 2025

Related U.S. Application Data
(60) Provisional application No. 63/477,024, filed on Dec. 23, 2022.

(51) Int. Cl. C12Q 1/6886

(52) U.S. Cl. CPC C12Q 1/6886; C12Q 2600/156; C12Q 2600/158

(57) ABSTRACT

Compositions and uses thereof for identification of hypomorphic p53 variants are provided. Also provided are methods of treating cancer and identification of subjects with increased risk of cancer.

### REFERENCE TO AN ELECTRONIC SEQUENCE LISTING

The contents of the electronic sequence listing labeled “WST-194.PCT.xml” (20,726 bytes and created Dec. 22, 2023) are herein incorporated by reference in their entirety.

### STATEMENT REGARDING FEDERALLY SPONSORED RESEARCH OR DEVELOPMENT

This invention was made with government support under CA102184 and CA238611 awarded by the National Institutes of Health. The government has certain rights in the invention.

### BACKGROUND

The p53 tumor suppressor gene plays a central role in cancer, as best illustrated by the fact that TP53 is mutated in over 50% of human tumors. Germline mutations in TP53 are present in families with Li Fraumeni syndrome (LFS), who develop multiple cancers of the brain, breast, bone, and adrenal cortex as early as the first decade of life. p53 also is a central player in the response of cells to genotoxic stress, and mutations in TP53 are associated with cancer therapy resistance and poor prognosis. Consistent with the latter premise, numerous studies have shown that missense mutant forms of p53 protein can possess oncogenic functions: these are referred to as “gain of function”. Prominent gain of function (GOF) activities by mutant p53 include the activation of SREBP1/2, growth factor receptor recycling, and activation of NF-1B signaling. It is important to note that despite widespread evidence for mutant p53 GOF, some tumor types do not show evidence for GOF. Typically, GOF activities of mutant p53 play roles in late-stage tumors, and GOF mutants are often associated with increased metastatic potential of tumors.

Unlike most tumor suppressor genes and oncogenes, there is significant germline coding region variation in TP53, with an estimated 200 germline missense variants in different ethnic groups. In some cases, these missense germline variants have been shown to impair but not completely inactivate p53 function; we refer to such variants as “hypomorphs”. We previously described one hypomorphic variant of TP53, Pro47Ser (P47S, rs1800371), which occurs in 1-2% of African-descent individuals, and is associated with increased cancer incidence in a mouse model and in humans. We showed that P47S protein is selectively defective in the ability to regulate p53 target genes associated with ferroptosis, or iron-lipid mediated cell death. This ferroptotic defect is correlated with increased production of glutathione and coenzyme A in P47S cells. The ferroptotic defect leads to iron accumulation in P47S mice; this led to our discovery that P47S is significantly associated with a disorder called Iron Overload in African Americans. Interestingly, the increase in glutathione in P47S cells leads to increased activation of mTOR due to increased Rheb-mTOR association; this leads to improved fitness and muscle recovery in P47S mice, suggesting that this allele may have been selected for at one time.

What is needed is improved compositions and methods for treatment of cancer.

### SUMMARY OF THE INVENTION

In one aspect, provided herein is a method of treating cancer in a subject in need thereof, the method comprising: a) obtaining a tumor sample from the subject; b) detecting expression levels of at least two genes in the tumor sample, wherein the genes are selected from: CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYBSA, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A, wherein a difference in the expression level of the genes as compared to a control level indicates the presence of a p53 hypomorphic variant; and c) administering a treatment for the cancer to the subject.

In another aspect, provided herein is a method of treating cancer in a subject in need thereof, the method comprising: having obtained information about expression levels of at least two genes in a sample obtained from the subject, wherein the genes are selected from: CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYBSA, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A, administering a treatment for the cancer to the subject, when a difference in the expression level of the genes as compared to a control level indicates the presence of a p53 hypomorphic variant.

In another aspect, provided herein is a method of reducing, inhibiting, and/or preventing tumor metastasis in a subject in need thereof, the method comprising: having obtained information about expression levels of two or more genes in a sample obtained from the subject, wherein the genes comprise CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYB5A, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A, wherein a difference in the expression level of the genes as compared to a control level indicates the presence of a p53 hypomorphic variant; and c) administering a treatment for the cancer to the subject.

In another aspect, provided herein is a method of assessing cancer risk in a subject, the method comprising: a) obtaining a biological sample from the subject; b) detecting expression levels of two or more genes in the sample, wherein the genes comprise: CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYBSA, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A, wherein a difference in the expression level of the genes as compared to a control level indicates an increased risk of cancer in the subject due to the presence of a p53 hypomorphic variant.

In another aspect, provided herein is a composition comprising a collection of probes, primers, and/or antibodies suitable for detection of the expression and/or expression levels of a collection of genes, or their products, in a tumor sample, wherein the collection of genes comprises CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYBSA, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3m, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A. In certain embodiments, a kit comprising the composition is provided.

Other aspects and advantages of the invention will be readily apparent from the following detailed description of the invention.

### DESCRIPTION OF THE FIGURES

FIG. 1A-FIG. 1I show p53 hypomorphs adopt a mutant conformation and express high levels of RRAD. (FIG. 1A) IPA analysis showing significance and effect on regulators with their targets significantly enriched among the genes differentially expressed in p53 hypomorphs P47S and Y107H. The bars represent activation Z-score of the regulators predicted by IPA based on direction of target changes, color represents significance p value, the number after the bar represents the number of targets affected. (FIG. 1B) Log₂ fold change plotted against −Log₁₀(p-value) for genes that respond to Nutlin-3a treatment after 24 hr in wild type. Highlighted pink are the genes that are activated to a greater extent in WT than in Y107H in response to treatment and labelled with names are the genes that behave similarly in P47S. The purple labels correspond to known p53 target genes. (FIG. 1C-FIG. 1E) LCL cell lines expressing hypomorphic p53, either (FIG. 1C) P47S-homozygous and heterozygous, (FIG. 1D) Y107H heterozygous, or (FIG. 1E) R273H heterozygous compared to regionally/family matched WT p53 LCL cell lines were treated with 10 μM Nutlin-3a for the indicated time points and harvested. Whole cell lysates were subjected to western blot analysis for the indicated antibodies. (FIG. 1F) WT, P47S, and Y107H HCT116 cells were analyzed for mutant p53 (pAb240) conformation by immunofluorescence analysis. Scale bar, 25 μm. (FIG. 1G) Quantification of the fraction of cells staining for mutant conformation (pAb240). Fractions indicated are such that 1.0=100%. Data are presented as mean±SD. n=25 random fields of view (>250 cells) from each of 2 independent experiments. ***p<0.001; #p<0.0001 by one-way ANOVA followed by Dunnett's multiple comparisons test. (FIG. 1H) Primary MEFs generated from WT, P47S, and Y107H mice were used to assess mutant p53 conformation by p53-Hsc70 interaction using PLA. Representative images are shown for each condition (scale bar, 20 m). White boxes denote the enlarged insets. (FIG. 1I) Quantification of the average number of PLA signals per cell. Values are expressed as mean±SD. n=6 random fields of view (>100 cells) from each of 2 independent experiments. ****p<0.0001 by one-way ANOVA followed by Dunnett's multiple comparisons test.

FIG. 2A-FIG. 2G show p53 hypomorphs exhibit elevated NF-κB signaling. (FIG. 2A-FIG. 2B) LCL cell lines expressing WT or hypomorphic p53, either (FIG. 2A) P47S or (FIG. 2B) Y107H were treated with 0.5 ng/mL TNF-α for the indicated periods. RNA was extracted and analyzed by Q-RT-PCR with primers specific for IL-6 and IL-8 mRNA. Values were normalized for GAPDH mRNA in the same sample. Values are expressed as mean±SD. Data are representative of n=6 technical replicates from 2 independent experiments. ****p<0.0001 by one-way ANOVA followed by Tukey's multiple comparisons test. (FIG. 2C) Representative immunohistochemical images of colon, spleen, thymus, and liver sections obtained from ten-week-old male mice of the indicated genotypes. Tissue sections were stained for phospho-p65 NF-κB (Ser276). Scale bar, 50 m. (FIG. 2D) Hsc70 immunoprecipitation in colon tissue harvested from ten-week-old male mice of the indicated genotypes. Immunoprecipitation of Hsc70 or IgG negative control was followed by immunoblotting for p53. (FIG. 2E) Whole cell lysates prepared from the colon and spleen of ten-week-old male mice of the indicated genotypes were analyzed by western blotting with the indicated antibodies. (FIG. 2F-FIG. 2G) WT and Y107H LCLs were treated with 0.5 ng/mL TNF-α for 0, 1, or 24 h and subjected to ChIP with (FIG. 2F) p65 NF-κB or (FIG. 2G) p53 antibodies, followed by Q-RT-PCR analysis with primers specific to the NF-κB site of the IL-6 and IL-8 promoters or a gene desert (negative control). Values are presented as percentage of input ±SD. The data represent three technical replicates and are representative of 2 biological replicates. *p<0.05; **p<0.01; ***p<0.001 as determined by two-tailed Student's t-test.

FIG. 3A-FIG. 3F show altered biochemical properties of hypomorphic p53 proteins. (FIG. 3A) WT, P47S, and Y107H LCLs were treated with or without 1 mM BMH for 30 minutes followed by immunoblotting for p53. (FIG. 3B) WT, P47S, and Y107H LCLs were treated with vehicle or 1 μg/mL ATO for 16 hr and then fixed with or without 1 mM BMH for 30 minutes followed by immunoblotting for p53. (FIG. 3C) WT or Y107H LCLs were untreated or treated with 10 μM Nutlin-3a (Nut), 1 μM doxorubicin (Dox), or 1 μg/mL arsenic trioxide (ATO) for 24 hr. Whole cell lysates were analyzed by western blotting with the indicated antibodies. (FIG. 3D) Cytosolic and nuclear fractions isolated from WT, P47S, and Y107H LCLs were analyzed by western blotting with the indicated antibodies. Histone H3 and GAPDH served as nuclear and cytosolic controls, respectively, to confirm efficiency of subcellular fractionation. (FIG. 3E-FIG. 3F) Chromatin-bound proteins were extracted from nuclear pellets isolated from WT, (FIG. 3E) P47S, and (FIG. 3F) Y107H LCLs using sequentially increasing salt concentrations (0 mM-500 mM NaCl) and analyzed by western blotting for p53.

FIG. 4A-FIG. 4E show p53 hypomorphs share a predictive gene signature. (FIG. 4A) Classification scores for training set samples obtained through leave one out cross-validation based on total 143 unique genes. Probability of 0.5 is used as the classification threshold to calculate accuracy. (FIG. 4B) Classification scores for independent test set. (FIG. 4C) IPA analysis showing enrichment and effect on regulators with targets significantly enriched among the 143 genes from the classifier. (FIG. 4D) Western blot analysis of WT, R175C, and Y220H LCLs treated with 10 μM Nutlin-3a for 24 hr. (FIG. 4E) Western blot analysis of WT, P47S, G360A, E11Q, and R110H LCLS treated with 10 μM Nutlin-3a for 24 h.

FIG. 5A-FIG. 5H show expression of p53 target genes and p53 protein conformation in hypomorphic cell lines. (FIG. 5A) Western blot analysis of WT or G334R LCLs treated with 10 μM Nutlin-3a for the indicated time points. (FIG. 5B) Western blot analysis of WT, P47S, and Y107H HCT116 cells treated with 10 μM Nutlin-3a for 24 hr. (FIG. 5C) Western blot analysis of WT, P47S, and Y107H primary MEFs treated with 5 μM CDDP for 24 hr. (FIG. 5D) WT, P47S, and Y107H HCT116 cells stained with the pan-p53 (1C12) antibody confirmed equivalent total p53 expression by immunofluorescence analysis. Scale bar, 25 m. (FIG. 5E) Representative images of single antibody (Hsp70) conditions for WT, P47S, and Y107H primary MEFs, which served as a negative control for proximity ligation assay analysis. Scale bar, 20 μm. (FIG. 5F) Western blot analysis of WT, P47S (homozygous), or R273H LCLs treated with 10 μM Nutlin-3a for the indicated time points. s.e., short exposure; i.e., long exposure. (FIG. 5G-FIG. 5H) Western blot analysis of WT, (FIG. 5G) P47S, and (FIG. 5H) Y107H LCLs treated with 10 μM Nutlin-3a for the indicated time points.

FIG. 6A-FIG. 6H show p53 hypomorph response to stimulation by EGF and TNF-α. (FIG. 6A-FIG. 6B) WT, (FIG. 6A) P47S, and (FIG. 6B) Y107H LCLs were treated with 0.1 g/mL EGF for the indicated time periods and analyzed by Q-RT-PCR with primers specific for fos and egfr mRNA. Values were normalized for GAPDH mRNA as an internal control. Data are presented as mean±SD. n=6 technical replicates from 2 independent experiments. *p<0.05; **p<0.01; ***p<0.001 by one-way ANOVA followed by Sidik's multiple comparisons test. (FIG. 6C) Hsc70 IP in thymus tissue harvested from 10-week-old male mice of the indicated genotypes. Immunoprecipitation of Hsc70 or IgG negative control was followed by immunoblotting for p53 (left). Whole cell lysates were analyzed by western blotting with the indicated antibodies (right). (FIG. 6D) Representative images of crystal violet staining following Matrigel chamber invasion assays with primary MEFs (scale bar, 100 μm). Cells were treated with 20 ng/mL TNF-α for 24 hr. Quantification of the average number of invaded cells per condition; values are expressed as mean±SD. n=5 random fields of view from each of 3 biological replicates. **p<0.01; ***p<0.001 as determined by two-tailed Student's t-test. (FIG. 6E) Representative images of crystal violet staining following Matrigel chamber invasion assays with HCT116 cells, treated with 20 ng/mL TNF-α for 30 hr (scale bar, 100 μm). Quantification of the average number of invaded cells per condition; values are expressed as mean±SD. n=5 random fields of view from each of 2 biological replicates. **p<0.01; #p<0.0001 as determined by two-tailed Student's t-test. (FIG. 6F-FIG. 6H) WT and Y107H LCLs were treated with 0.5 ng/mL TNF-α for 0, 1, or 24 h and subjected to ChIP with (FIG. 6F-FIG. 6G) IgG, H3K27ac, or (FIG. 6H) p53 antibodies, followed by Q-RT-PCR analysis with primers specific to the NF-κB site of the (FIG. 6F) IL-6 and (FIG. 6G) IL-8 promoters, as well as the (FIG. 6H) p53 site of the p21 promoter. Values are presented as percentage of input. Error bars represent ±SD. *p<0.05; **p<0.01; ***p<0.001 as determined by two-tailed Student's t-test.

FIG. 7A-FIG. 7C show predictive p53 hypomorph gene signature and RRAD expression in benign and presumed hypomorphic p53 cell lines. (FIG. 7A) ROC AUC for FIG. 7A. (FIG. 7B) ROC AUC for FIG. 4B. (FIG. 7C) Western blot analysis of WT, P47S, or R110H LCLs treated with 10 μM Nutlin-3a for 24 hr.

FIG. 8A-FIG. 8H show P47S mice are sensitive to DSS-induced chronic inflammation. (FIG. 8A) Schematic detailing the treatment schedule for inducing chronic inflammation in WT, P47S, and Y107H mice by administering three cycles of DSS. (FIG. 8B) Mouse weights recorded throughout the study for both untreated mice and mice exposed to 2% DSS for each genotype. (FIG. 8C-FIG. 8D) At the conclusion of the study, (FIG. 8C) spleen weight and (FIG. 8D) colon lengths were measured as proximal analyses of inflammation response to DSS treatment. (FIG. 8E-FIG. 8F) Colonic inflammation was assessed by histopathological analysis; (FIG. 8E) total inflammation score and (FIG. 8F) hyperplasia atypia scores are reported. (FIG. 8G-FIG. 8H) Representative images of colon tissues in P47S mice demonstrating observed instances of (FIG. 8G) reactive hyperplasia reminiscent of colitis profunda (asterisk), as well as (FIG. 8H) extension into the submucosa (asterisk) and fibrotic reaction (arrow; higher magnification image, right). n=6-8 mice per gender per treatment group; all analyses represent pooled data for both male and female mice.

### DETAILED DESCRIPTION OF THE INVENTION

There is considerable genetic diversity in the TP53 gene, with hundreds of missense single nucleotide polymorphisms (SNPs) that may alter p53 function. While cancer-causing TP53 mutations are well characterized, many of the germline missense variants remain unclassified because their impact on p53 function and cancer risk is unknown.

We describe herein that the hypomorphic p53 variants, including P47S and Y107H, despite being located in different domains of p53, both have increased propensity to misfold compared to WT p53. Moreover, we determined that these variants adopt a protein conformation commonly associated with tumor-derived mutant forms of p53. Mutant p53 is known to enhance NF-κB activity, and we show that the P47S and Y107H hypomorphs are both associated with increased NF-κB activity in unstressed human cells and mouse tissues. We then used machine-learning approaches on RNA Sequencing data from unstressed lymphoblastoid cells to identify a gene signature that accurately distinguishes p53 hypomorphs from WT p53 and even a benign variant. The findings demonstrate, inter alia, that we are able to broadly screen individuals with genetic variants of p53, and better inform them of their cancer risk and select appropriate treatment regimes.

It is to be noted that the term “a” or “an” as used herein refers to one or more. As such, the terms “a” (or “an”), “one or more,” and “at least one” are used interchangeably herein.

While various embodiments in the specification are presented using “comprising” language, under other circumstances, a related embodiment is also intended to be interpreted and described using “consisting of” or “consisting essentially of” language. The words “comprise”, “comprises”, and “comprising” are to be interpreted inclusively rather than exclusively. The words “consist”, “consisting”, and its variants, are to be interpreted exclusively, rather than inclusively.

As used herein, the term “about” means a variability of 10% from the reference given, unless otherwise specified.

“Upregulation” as used herein refers to an elevation in the level of expression of a product of one or more genes in a cell or the cells of a tissue or organ.

“Downregulate” as used herein refers to a reduction in the level of expression of a product of one or more genes in a cell or the cells of a tissue or organ.

A “subject” is a mammal, e.g., a human, mouse, rat, guinea pig, dog, cat, horse, cow, pig, or non-human primate, such as a monkey, chimpanzee, baboon, or gorilla. The term “patient” may be used interchangeably with the term subject. In one embodiment, the subject is a human. The subject may be of any age, as determined by the health care provider. In certain embodiments described herein, the patient is a subject who has previously been diagnosed with cancer. The subject may have been treated for cancer previously, or is currently being treated for cancer. In one embodiment, the subject is experiencing stress which has an impact on the beta-adrenergic signaling pathway.

“Sample” as used herein means any biological fluid or tissue that contains blood cells, immune cells, and/or cancer cells. In one embodiment, the sample is whole blood or peripheral blood mononuclear cells (PBMC). Other useful biological samples include, without limitation, plasma, saliva, synovial fluid, bone marrow, cerebrospinal fluid, vaginal mucus, cervical mucus, nasal secretions, sputum, semen, amniotic fluid, bronchoscopy sample, bronchoalveolar lavage fluid, and other cellular exudates from a patient having cancer. Such samples may further be diluted with saline, buffer or a physiologically acceptable diluent. Alternatively, such samples are concentrated by conventional means. In certain embodiments, the sample, or cells in the sample, are not subject to any additional stresses, once removed from the subject.

The term “cancer” or “proliferative disease” as used herein means any disease, condition, trait, genotype, or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive and is also referred to as a neoplasm. Tumors can be either benign (not cancerous) or malignant. The methods described herein are useful for the treatment of cancer and tumor cells, i.e., both malignant and benign tumors. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multi-drug resistant cancers. In one embodiment, the cancer is lung cancer. In another embodiment, the cancer is ovarian cancer. In another embodiment, the cancer is prostate cancer. In another embodiment, the cancer is lung cancer. In another embodiment, the cancer is colorectal cancer. In another embodiment, the cancer is brain cancer. In certain embodiments, the cancer is pancreatic ductal adenocarcinoma (PDAC). In certain embodiments, the cancer is glioblastoma (GBM).

“Control” or “control level” as used herein refers to the source of the reference value for the biomarker gene levels as well as the particular panel of genes in a control obtained from cells or tissues that lack a p53 hypomorphic variant. In certain embodiments, the control levels are obtained from cells or tissues that have at least one TP53 wildtype allele. In certain embodiments, the control levels are obtained from cells or tissues that have a benign TP53 variant allele. In another embodiment, the control or reference level is from a population of individuals sharing a specific p53 variant. In yet another embodiment, the control or reference level is an assigned value which correlates with the level of a specific control individual or population, although not necessarily measured at the time of assaying the test subject's sample. Selection of the particular class of controls depends upon the use to which the diagnostic/monitoring methods and compositions are to be put by the physician. In certain embodiments, the control or control level is obtained from healthy or normal tissue in a subject with disease. In certain embodiments, the control or control level is obtained from healthy or normal tissue in a subject without disease. In certain embodiments, the control or control level is obtained from a tumor-free margin.

The term “hypomorph” and “hypermorphic” as used herein refers to a p53 protein that is non-functional or lesser-functioning than wild type p53, or a variant TP53 coding sequence that encodes the non-functional or lesser-functioning p53 protein. For example, in some instances, variation in the TP53 coding sequence results in expression a hypomorphic protein that is defective due to misfolding or truncation. Cells and tissues containing these hypomorphic variants may also show increased NF-κB activity or increased levels of cell migration, invasion, and/or metastasis. In certain embodiments, the methods and compositions provided herein are useful in distinguishing p53 hypomorphs that increase cancer risk from benign variants and/or wild type p53. Examples of hypomorphic variants include P47S (Pro47Ser, rs1800371), Y107H (Tyr107His, rs368771578), or G334R (Gly334Arg, rs78378222). Still others are described in the literature, See e.g., Kato et al. Proc Natl Acad Sci USA. 2003 Jul. 8; 100(14):8424-9; Giacomelli et al. Nat Genet. 2018 October; 50(10): 1381-1387; and Kotler et al. Mol Cell. 2018 Sep. 6; 71(5):873, which are incorporated herein by reference.

Missense mutations that inactivate p53 occur commonly in cancer, and germline mutations in TP53 cause Li Fraumeni syndrome, which is associated with early-onset cancer. In addition, there are over two hundred germline missense variants of p53 that remain uncharacterized. In some cases, these germline variants have been shown to encode a hypomorphic p53 protein, and these alleles are associated with increased cancer risk in humans and mouse models. However, the majority of p53 variants remain un- or mis-classified in clinical genetics databases.

Recently some groups have attempted to establish the functionality of p53 variants in non-transformed cell lines containing endogenous p53 variants, following exposure to genotoxic stress. However, such studies have been low throughput, as they require a genotoxic stress, and their data interpretation may be confounded by the existence of other variants in the p53 pathway that influence the transcriptional response, such as SNP309 in MDM2. Herein, provided is a gene signature for a p53 hypomorph from unstressed cells. The compositions and their uses have the potential to markedly impact the categorization of these variants, and to better inform carriers of cancer risk, and the optimal choices for cancer therapy.

By “fragment” is intended a molecule consisting of only a part of the intact full-length polypeptide sequence and structure. The fragment can include a C terminal deletion, an N terminal deletion, and/or an internal deletion of the native polypeptide. A fragment will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence, provided that the fragment in question retains the ability to elicit the desired biological response, although not necessarily at the same level.

By the term “antibody” or “antibody molecule” is meant any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof. An antibody fragment can be a synthetically or genetically engineered polypeptide. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment, which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

The term “derived from” is used to identify the original source of a molecule (e.g., bovine or human) but is not meant to limit the method by which the molecule is made which can be, for example, by chemical synthesis or recombinant means.

By “change in expression” is meant an upregulation of one or more selected genes in comparison to the reference or control; a downregulation of one or more selected genes in comparison to the reference or control; or a combination of certain upregulated genes and down regulated genes.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc.

By “target biomarker” or “target biomarker signature” as used herein is meant those proteins/peptides or the genes/transcripts encoding same, the expression of which changes (either in an up-regulated or down-regulated manner) characteristically in the presence of a p53 hypomorph. In certain embodiments, at least two target biomarkers form a suitable biomarker signature for use in the methods and compositions. In certain embodiments, at least three target biomarkers form a suitable biomarker signature for use in the methods and compositions. In certain embodiments, at least four target biomarkers form a suitable biomarker signature for use in the methods and compositions. In certain embodiments, at least five biomarkers form a suitable biomarker signature for use in the methods and compositions. In one embodiment, at least six target biomarkers form a suitable biomarker signature for use in the methods and compositions. In certain embodiments, at least seven target biomarkers form a suitable biomarker signature for use in the methods and compositions. In certain embodiments, at least eight target biomarkers form a suitable biomarker signature for use in the methods and compositions. In certain embodiments, at least nine target biomarkers form a suitable biomarker signature for use in the methods and compositions. In certain embodiments, at least ten target biomarkers form a suitable biomarker signature for use in the methods and compositions. In certain embodiments, at least ten target biomarkers form a suitable biomarker signature for use in the methods and compositions. In still further embodiments, at least 20, at least 30, at least 40, at least 50 or at least 60 of the biomarkers including any numbers therebetween identified in Table 1 form a suitable biomarker signature for the identification of a p53 hypomorph. Specific biomarker signatures can include any combination of p53 hypomorph biomarkers employing at least two biomarkers identified in Table 1 or including all 143 biomarkers in Table 1. One skilled in the art may readily reproduce the compositions and methods described herein by use of the sequences of the biomarkers, all of which are publicly available from conventional sources, such as GenBank.

TABLE 1

| EnsembleID | Symbol |
|---|---|
| ENSG00000076650 | GPATCH1 |
| ENSG00000068097 | HEATR6 |
| ENSG00000100028 | SNRPD3 |
| ENSG00000100412 | ACO2 |
| ENSG00000102265 | TIMP1 |
| ENSG00000104490 | NCALD |
| ENSG00000109861 | CTSC |
| ENSG00000110841 | PPFIBP1 |
| ENSG00000111729 | CLEC4A |
| ENSG00000010810 | FYN |
| ENSG00000119397 | CNTRL |
| ENSG00000121579 | NAA50 |
| ENSG00000122642 | FKBP9 |
| ENSG00000122862 | SRGN |
| ENSG00000123892 | RAB38 |
| ENSG00000124766 | SOX4 |
| ENSG00000125450 | NUP85 |
| ENSG00000125657 | TNFSF9 |
| ENSG00000126262 | FFAR2 |
| ENSG00000126767 | ELK1 |
| ENSG00000126787 | DLGAP5 |
| ENSG00000127666 | TICAM1 |
| ENSG00000100227 | POLDIP3 |
| ENSG00000130303 | BST2 |
| ENSG00000130513 | GDF15 |
| ENSG00000089280 | FUS |
| ENSG00000134590 | FAM127A |
| ENSG00000134644 | PUM1 |
| ENSG00000135597 | REPS1 |
| ENSG00000135838 | NPL |
| ENSG00000111913 | FAM65B |
| ENSG00000083937 | CHMP2B |
| ENSG00000109220 | CHIC2 |
| ENSG00000080819 | CPOX |
| ENSG00000115539 | PDCL3 |
| ENSG00000059728 | MXD1 |
| ENSG00000075785 | RAB7A |
| ENSG00000038427 | VCAN |
| ENSG00000120727 | PAIP2 |
| ENSG00000041802 | LSG1 |
| ENSG00000119048 | UBE2B |
| ENSG00000029534 | ANK1 |
| ENSG00000075239 | ACAT1 |
| ENSG00000139192 | TAPBPL |
| ENSG00000110934 | BIN2 |
| ENSG00000140263 | SORD |
| ENSG00000051180 | RAD51 |
| ENSG00000140374 | ETFA |
| ENSG00000143195 | ILDR2 |
| ENSG00000144655 | CSRNP1 |
| ENSG00000145779 | TNFAIP8 |
| ENSG00000120889 | TNFRSF10B |
| ENSG00000150540 | HNMT |
| ENSG00000152601 | MBNL1 |
| ENSG00000154760 | SLFN13 |
| ENSG00000172530 | BANP |
| ENSG00000156535 | CD109 |
| ENSG00000157782 | CABP1 |
| ENSG00000160213 | CSTB |
| ENSG00000166401 | SERPINB8 |
| ENSG00000164086 | DUSP7 |
| ENSG00000164124 | TMEM144 |
| ENSG00000164211 | STARD4 |
| ENSG00000164603 | C7orf60 |
| ENSG00000165271 | NOL6 |
| ENSG00000122376 | FAM35A |
| ENSG00000102893 | PHKB |
| ENSG00000166347 | CYB5A |
| ENSG00000205544 | TMEM256 |
| ENSG00000169129 | AFAP1L2 |
| ENSG00000171723 | GPHN |
| ENSG00000250565 | ATP6V1E2 |
| ENSG00000169245 | CXCL10 |
| ENSG00000170027 | YWHAG |
| ENSG00000173548 | SNX33 |
| ENSG00000066136 | NFYC |
| ENSG00000118518 | RNF146 |
| ENSG00000015171 | ZMYND11 |
| ENSG00000138777 | PPA2 |
| ENSG00000173801 | JUP |
| ENSG00000167851 | CD300A |
| ENSG00000120029 | C10orf76 |
| ENSG00000059769 | DNAJC25 |
| ENSG00000180353 | HCLS1 |
| ENSG00000173826 | KCNH6 |
| ENSG00000023892 | DEF6 |
| ENSG00000163687 | DNASE1L3 |
| ENSG00000076382 | SPAG5 |
| ENSG00000176381 | PRR18 |
| ENSG00000007237 | GAS7 |
| ENSG00000177453 | NIM1K |
| ENSG00000166949 | SMAD3 |
| ENSG00000214517 | PPME1 |
| ENSG00000184867 | ARMCX2 |
| ENSG00000184371 | CSF1 |
| ENSG00000185010 | F8 |
| ENSG00000183621 | ZNF438 |
| ENSG00000087086 | FTL |
| ENSG00000184445 | KNTC1 |
| ENSG00000166352 | C11orf74 |
| ENSG00000146278 | PNRC1 |
| ENSG00000188372 | ZP3 |
| ENSG00000178498 | DTX3 |
| ENSG00000169813 | HNRNPF |
| ENSG00000176986 | SEC24C |
| ENSG00000075651 | PLD1 |
| ENSG00000152779 | SLC16A12 |
| ENSG00000196131 | VN1R2 |
| ENSG00000204248 | COL11A2 |
| ENSG00000132676 | DAP3 |
| ENSG00000132128 | LRRC41 |
| ENSG00000126561 | STAT5A |
| ENSG00000177105 | RHOG |
| ENSG00000100243 | CYB5R3 |
| ENSG00000116560 | SFPQ |
| ENSG00000198113 | TOR4A |
| ENSG00000088888 | MAVS |
| ENSG00000100101 | NOL12 |
| ENSG00000122786 | CALD1 |
| ENSG00000185619 | PCGF3 |
| ENSG00000135750 | KCNK1 |
| ENSG00000117153 | KLHL12 |
| ENSG00000148773 | MKI67 |
| ENSG00000116815 | CD58 |
| ENSG00000116171 | SCP2 |
| ENSG00000158470 | B4GALT5 |
| ENSG00000162368 | CMPK1 |
| ENSG00000072401 | UBE2D1 |
| ENSG00000106780 | MEGF9 |
| ENSG00000204304 | PBX2 |
| ENSG00000117115 | PADI2 |
| ENSG00000065526 | SPEN |
| ENSG00000165997 | ARL5B |
| ENSG00000125821 | DTD1 |
| ENSG00000133119 | RFC3 |
| ENSG00000139618 | BRCA2 |
| ENSG00000206527 | PTPLB |
| ENSG00000143669 | LYST |
| ENSG00000144597 | EAF1 |
| ENSG00000214309 | MBLAC1 |
| ENSG00000226025 | LGALS17A |
| ENSG00000169136 | ATF5 |
| ENSG00000032389 | TSSC1 |

In certain embodiments, upregulation of two or more target biomarkers forms a suitable biomarker signature for use in the methods and compositions. In certain embodiments, the upregulated target biomarkers include two or more of CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYBSA, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, and DAP3. In certain embodiments, the upregulation of this collection of genes forms a suitable biomarker signature for use in the methods and compositions.

In certain embodiments, downregulation of two or more target biomarkers forms a suitable biomarker signature for use in the methods and compositions. In certain embodiments, the downregulated target biomarkers include two or more of POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A. In certain embodiments, the downregulation of this collection of genes forms a suitable biomarker signature for use in the methods and compositions.

The term “microarray” refers to an ordered arrangement of hybridizable array elements, e.g., primers, probes, ligands, on a substrate.

The term “ligand” refers to a molecule that binds to a protein or peptide, and includes antibodies and fragments thereof.

The term “polynucleotide,” when used in singular or plural form, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, “labels” or “reporter molecules” are chemical or biochemical moieties useful for labeling a nucleic acid (including a single nucleotide), polynucleotide, oligonucleotide, or protein ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to an oligonucleotide or nucleotide (e.g., a non-natural nucleotide) or ligand.

As used herein, the term “a therapeutically effective amount” refers an amount sufficient to achieve the intended purpose. For example, an effective amount of chemotherapeutic agent to kill p53 hypomorphic cancer cells. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined by a skilled artisan according to established methods in the art.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

Routes of administration include, but are not limited to, intradermal, intramuscular, intraperitoneal, intravenous, subcutaneous, intranasal, epidural, and oral routes. The agent may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As used herein, “disease”, “disorder”, and “condition” are used interchangeably, to indicate an abnormal state in a subject.

### Target Biomarkers and Biomarker Signatures Useful in the Methods and Compositions

Mutational inactivation of TP53 occurs commonly in cancer. In addition to mutation, there are over two hundred germline genetic variants in the TP53 coding region; some of these produce partially functional (hypomorphic) protein. Provided herein, is an optimized gene signature for identification of TP53 hypomorphs. The “targets” of the compositions and methods of these inventions include, in one aspect, the genes, gene fragments, transcripts, and the expression products, including the proteins and fragments thereof, listed in Table 1, which comprise this gene signature. As described in the Examples below, the inventors identified a collection of genes that distinguish p53 hypomorphs from wildtype p53. In certain embodiments, provided herein are superior tests for detection of p53 hypomorphs utilizing at least two of the biomarkers to provide more effective detection of cancer or cancer risk in a subject. In other embodiments, superior diagnostic tests utilizing at least two of the biomarkers for evaluating the prognosis of cancer treatment utilize are provided. In other embodiments, the methods utilize at least 3, 4, 5, 6, 7, 8, 9, 10, 11, 12, 13, 14, 15, 20, 30, 40, 50, 60, or more of the specific target biomarkers identified herein. In certain embodiments, the method employs all 143 of the biomarkers identified in Table 1.

### Diagnostic Reagents and Kits

### Labeled or Immobilized Biomarkers or Peptides

In certain embodiments, diagnostic reagents for use in methods of identifying a p53 hypomorph in a sample include at least two target biomarkers identified in Table 1 herein, associated with a detectable label or portion of a detectable label system. In certain embodiments, a diagnostic reagent includes at least two target biomarkers identified in Table 1 immobilized on a substrate. In still another embodiment, combinations of such labeled or immobilized biomarkers are suitable reagents and components of a diagnostic kit. Among such immobilized or labeled biomarkers are those selected from the biomarkers: CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYBSA, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A.

In certain embodiments, suitable embodiments of such labeled or immobilized reagents detect at least 2, 3, 4, 5, 6, 7, 8, 9, 10, 11, 12, 13, 14, 15, 20, 30, 40, 50, or 60 of the biomarkers in Table 1. In another aspect, suitable embodiments of such labeled or immobilized reagents detect all 143 of the biomarkers in Table 1.

Any combination of labeled or immobilized biomarkers can be assembled in a kit for the purposes of identifying a p53 hypomorph in cells or tissues. For example, one embodiment of a kit includes labeled or immobilized reagents for detection of the 143 biomarkers in Table 1.

For these reagents, the labels may be selected from among many known diagnostic labels, including those described above. Similarly, the substrates for immobilization may be any of the common substrates, glass, plastic, a microarray, a microfluidics card, a chip or a chamber.

Labeled or Immobilized Ligands that Bind the Biomarkers or Peptides

In curtain embodiments, the diagnostic reagent includes ligands that bind to biomarkers, or a fragment thereof, as listed in Table 1. Such a ligand desirably binds to a protein biomarker or a fragment thereof, and can be an antibody which specifically binds a single biomarker from Table 1. Various forms of antibody, e.g., polyclonal, monoclonal, recombinant, chimeric, as well as fragments and components (e.g., CDRs, single chain variable regions, etc.) may be used in place of antibodies. The ligand itself may be labeled or immobilized.

In certain embodiments, suitable embodiments of such labeled or immobilized reagents detect at least 2, 3, 4, 5, 6, 7, 8, 9, 10, 11, 12, 13, 14, 15, 20, 30, 40, 50, or 60 of the ligands. Each ligand binds to a single biomarker listed in Table 1. In certain embodiments, such labeled or immobilized reagents include ligands that bind all 143 biomarkers listed in Table 1.

Any combination of labeled or immobilized biomarker-binding ligands can be assembled in a kit for the purposes of identifying a p53 hypomorph in cells or tissues. For example, one embodiment of a kit includes labeled or immobilized reagents that bind to biomarkers listed in Table 1.

Labeled or Immobilized Polynucleotide Oligonucleotides that Hybridize to Genes, Gene Fragments, Gene Transcripts of other Sequences Encoding the Biomarkers or Peptides

In another embodiment, the diagnostic reagent includes polynucleotide or oligonucleotide sequences that hybridize to genes, gene fragments, gene transcripts or nucleotide sequences encoding the biomarkers listed in Table 1. Such a polynucleotide/oligonucleotide can be a probe or primer, and may itself be labeled or immobilized. In another aspect, suitable embodiments of such labeled or immobilized reagents include least 2, 3, 4, 5, 6, 7, 8, 9, 10, 11, 12, 13, 14, 15, 20, 30, 40, 50, or 60 polynucleotides/oligonucleotides. Each polynucleotide/oligonucleotide hybridizes to a gene, gene fragment, gene transcript or expression product encoding a single biomarker listed in Table 1. Any combination of labeled or immobilized biomarker-hybridizable sequences can be assembled in a kit for the purposes of identifying a gene signature indicative of a p53 hypomorph. For example, certain embodiments of a kit include labeled or immobilized reagents that hybridize to all 143 biomarkers listed in Table 1.

Still other components of the many biomarker signatures that may be formed by various combinations of polynucleotide/oligonucleotide sequences that hybridize to the biomarkers listed in Table 1 associated with detectable labels or immobilized on substrates provide additional diagnostic kits. Still other components include similar reagents that hybridize to biomarkers or fragments thereof as listed in Table 1. In certain embodiments, these polynucleotide or oligonucleotide reagent(s) are part of a primer-probe set, and the kit comprises both primers and probes. Each said primer-probe set amplifies a different gene, gene fragment or gene expression product that encodes a different biomarker of any combination of the biomarkers listened in Table 1.

The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publicly available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the web for general users and for biologist programmers and other publications).

Thus, a composition for identifying a p53 hypomorph in cells or tissues in a mammalian subject as described herein can be a kit containing multiple reagents or one or more individual reagents. For example, one embodiment of a composition includes a substrate upon which the biomarkers, polynucleotides or oligonucleotides, or ligands are immobilized. In another embodiment, the composition is a kit also contains optional detectable labels, immobilization substrates, optional substrates for enzymatic labels, as well as other laboratory items.

The compositions based on the biomarkers selected from Table 1 described herein, optionally associated with detectable labels, can be presented in the format of a microfluidics card, a chip or chamber, or a kit adapted for use with the assays described in the Examples.

The selection of the ligands, poly/oligonucleotide sequences, their length, suitable labels and substrates used in the composition are routine determinations made by one of skill in the art in view of the teachings of which biomarkers form signature suitable for identifying a p53 hypomorph in cells or tissues.

Methods for Diagnosing, Treating, and/or Assessing Cancer Risk

### Protein Assays

In certain embodiments, a method for identifying a p53 hypomorph in cells or tissues from a subject includes measuring in a biological fluid sample of the subject the expression level of a protein or fragment thereof selected from at least one biomarker of Table 1. Alternatively, the method includes measuring a combination of at least 2, 3, 4, 5, 6, 7, 8, 9, or 10, 20, 30, 40, 50, 60, 70, 80, 90, 100, 110, 120, 130, or 140 biomarkers in Table 1. In certain embodiments, the method includes measuring a combination of all 143 biomarkers of Table 1. The method involves comparing the expression level of the selected biomarker or biomarker fragment with the level of the same protein or peptide in the biological fluid of a reference or control mammalian subject. Differences in expression of the subject's selected biomarker protein or peptide fragment from those of the reference or control correlates with the presence of a p53 hypomorph. In certain embodiments, differences in expression of the subject's selected biomarker protein or peptide fragment from those of the reference or control correlates with shortened patient survival. In certain embodiments, differences in expression of the subject's selected biomarker protein or peptide fragment from those of the reference or control correlates with cancer treatment failure.

In this method, a difference in expression level of one or more of the selected biomarker proteins in comparison to the control reference may be an increase or decrease in the expression levels of the individual biomarkers. This method may employ any of the suitable diagnostic reagents or kits or compositions described above.

The measurement of the biomarkers in the biological sample may employ any suitable ligand, e.g., antibody (or antibody to any second biomarker) to detect the biomarker protein. Such antibodies may be presently extant in the art or presently used commercially, such as those available as part of commercial antibody ELISA assay kits or that may be developed by techniques now common in the field.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The biomarker-antibodies or fragments useful in this invention are not limited by the particular detectable label or label system employed. Thus, selection and/or generation of suitable biomarker-antibodies with optional labels for use in this invention is within the skill of the art, provided with this specification, the documents incorporated herein, and the conventional teachings of immunology.

Similarly, the particular assay format used to measure the selected biomarker in a biological sample may be selected from among a wide range of immunoassays, such as enzyme-linked immunoassays, such as those described in the examples below, sandwich immunoassays, homogeneous assays, immunohistochemistry formats, or other conventional assay formats. One of skill in the art may readily select from any number of conventional immunoassay formats to perform this invention.

Other reagents for the detection of protein in biological samples, such as peptide mimetics, synthetic chemical compounds capable of detecting the selected EP biomarker may be used in other assay formats for the quantitative detection of biomarker protein in biological samples, such as high-pressure liquid chromatography (HPLC), immunohistochemistry, etc.

### Nucleic Acid Assays

Still other methods useful in performing the diagnostic steps described herein are known in the art. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as real-time polymerase chain reaction (RT-PCR), qPCR, and quantitative RT-PCR (Q-RT-PCR) (e.g., using the QuantStudio 5 Real-Time PCR System (Applied Biosystems)). Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, CA), differential display, amplified fragment length polymorphism (iAFLP), and BeadArray™ technology (Illumina, San Diego, CA) using the commercially available Luminex100 LabMAP system and multiple color-coded microspheres (Luminex Corp., Austin, Tex.) and high coverage expression profiling (HiCEP) analysis.

Thus, in certain embodiments, a method for treating cancer is provided that includes obtaining a tumor sample from the subject detecting expression levels of at least two genes in the tumor sample, wherein the genes are selected from the biomarkers listed in Table 1. In other embodiments, a method of reducing, inhibiting, and/or preventing tumor metastasis in a subject in need thereof is provided that includes detecting the expression levels of biomarkers listed in Table 1. In yet another aspect, a method for assessing cancer risk in a subject in a subject is provided that includes obtaining a biological sample from the subject and detecting the expression levels of biomarkers listed in Table 1. For example, in certain embodiments, the biological sample includes primary peripheral blood mononuclear cells. These methods may employ any of the suitable diagnostic reagents or kits or compositions described above.

In certain embodiments, an increase in expression level of one or more of the selected biomarker genes, gene fragments, gene transcripts or expression products in comparison to a control reference is detected. In certain embodiments, an increase in expression level of all of the selected biomarker genes, gene fragments, gene transcripts or expression products in comparison to a control reference is detected. In certain embodiments, an increase in expression level of one or more of the selected biomarker genes, gene fragments, gene transcripts or expression products in comparison to a control reference is greater than or equal to 1.1 fold, 1.2 fold, 1.3 fold, 1.4 fold, 1.5 fold, 1.75 fold, 2.0 fold, 4.0 fold, 5.0 fold, 6.0 fold, 8.0 fold, or 10 fold. In certain embodiments, a decrease in expression level of one or more of the selected biomarker genes, gene fragments, gene transcripts or expression products in comparison to a control reference is greater than or equal to 1.1 fold, 1.2 fold, 1.3 fold, 1.4 fold, 1.5 fold, 1.75 fold, 2.0 fold, 4.0 fold, 5.0 fold, 6.0 fold, 8.0 fold, or 10 fold.

In yet another embodiment, the methods and compositions described herein may be used in conjunction with clinical risk factors to help physicians make more accurate decisions about how to manage patients with cancers with p53 hypomorphic variants. Another advantage of these methods and compositions is that diagnosis may occur earlier and treatment may commence earlier. In certain embodiments, the treatment includes administering a chemotherapy, radiation therapy, immunotherapy, hormone therapy, stem cell or bone marrow transplant, and/or surgical resection.

Chemotherapeutic agents (e.g., anti-cancer agents) are well known in the art and include, but are not limited to, anthracenediones (anthraquinones) such as anthracyclines (e.g., daunorubicin (daunomycin; rubidomycin), doxorubicin, epirubicin, idarubicin, and valrubicin), mitoxantrone, and pixantrone; platinum-based agents (e.g., cisplatin, carboplatin, oxaliplatin, satraplatin, picoplatin, nedaplatin, triplatin, and lipoplatin); tamoxifen and metabolites thereof such as 4-hydroxytamoxifen (afimoxifene) and N-desmethyl-4-hydroxytamoxifen (endoxifen); taxanes such as paclitaxel (taxol) and docetaxel; alkylating agents (e.g., nitrogen mustards such as mechlorethamine (HN2), cyclophosphamide, ifosfamide, melphalan (L-sarcolysin), and chlorambucil); ethylenimines and methylmelamines (e.g., hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazenoimidazolecarboxamide)); antimetabolites (e.g., folic acid analogues such as methotrexate (amethopterin), pyrimidine analogues such as fluorouracil (5-fluorouracil; 5-FU), floxuridine (fluorodeoxyuridine; FUdR), and cytarabine (cytosine arabinoside), and purine analogues and related inhibitors such as mercaptopurine (6-mercaptopurine; 6-MP), thioguanine (6-thioguanine; 6-TG), and pentostatin (2′-deoxycofonnycin)); natural products (e.g., vinca alkaloids such as vinblastine (VLB) and vincristine, epipodophyllotoxins such as etoposide and teniposide, and antibiotics such as dactinomycin (actinomycin D), bleomycin, plicamycin (mithramycin), and mitomycin (mitomycin Q); enzymes such as L-asparaginase; biological response modifiers such as interferon alpha); substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof derivatives thereof and combinations thereof.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### EXAMPLES

### Example 1: Materials and Methods

### Materials and Methods

Mammalian cell culture. Human EBV-immortalized lymphoblastoid (LCL) cell lines were obtained from the Coriell Institute (Camden, New Jersey). LCLs were cultured in RPMI 1640 supplemented with 15% fetal bovine serum, 1% penicillin/streptomycin, and 2 mM L-glutamine. Primary and transformed mouse embryonic fibroblasts (MEFs) were cultured in DMEM supplemented with 10% fetal bovine serum and 1% penicillin/streptomycin. Cells were grown in a 5% CO2 humidified incubator at 37 °C. All cell lines were confirmed to be free of mycoplasma prior to each experiment. WT, P47S, and Y107H primary mouse embryonic fibroblasts (MEF) were isolated from 13.5-day-old Hupki embryos and cultured at 37 °C in DMEM with 1% penicillin/streptomycin and 10% FBS.

For the HCT116 P47S and Y107H cell lines, the P47S and Y107H point mutations were introduced respectively by nucleofection of HCT116 cells with a synthetic gRNA/Cas9 ribonucleoprotein complex along with a ssODN. The gRNA recognition site for P47S is 5′-ACCATTGTTCAATATCGTCCNGG (SEQ ID NO: 1), and the ssODN has the following sequence with two phosphorothioate bonds at each end: 5′cttttcacccatctacagtcccccttgccgtcccaagcaatggatgatttgatgctgtccAGTgacgatattgaacaatggttcactg aagacccaggtccagatgaagctcccagaatg (SEQ ID NO: 2). The gRNA recognition site for Y107H is 5′-aaaacctaccagggcagctaCGG (SEQ ID NO: 3), with the PAM site in upper case, and the ssODN has the following sequence with two phosphorothioate bonds at each end: 5′-tgaccgtgcaagtcacagacttggctgtcccagaatgcaagaagcccagacggaaaccgtGGGAgccctggtaggttttctggga agggacagaagatgacaggggccaggagggggctggtgc (SEQ ID NO: 4). Both synthetic gRNAs and ssODNs in ultramer format were purchased from IDT. The transfected pools of HCT 116 cells were analyzed by using Next Generation Sequencing for knock-in rate, and single-cell clones were obtained by sorting on a Sony sorter and screened by using Next Gen Sequencing. Positive clones were expanded, genotype confirmed prior to cryopreservation. All clones are negative for mycoplasma contamination and authenticated as HCT116 cells by STR profiling. P47S clones B5 (cl1) and G12 (cl2) and Y107H clones All (cl1) and C2 (cl2) were confirmed and the TP53 cDNA was sequenced in its entirety. Cells were treated with 10 μM Nutlin-3a (Sigma-Aldrich) or 5 μM CDDP (cisplatin; Med Chem Express) where indicated to induce p53 signaling. To assess NF-κB signaling, cells were treated with TNF-α (Sigma-Aldrich) at the indicated concentrations and time points. Prior to treatment with EGF (Sigma-Adrich), cells were serum starved in media supplemented with 1% fetal bovine serum for 6 hr.

TABLE 2
B lymphoblastoid cell lines

| Cell Line/Classification | Source | Classifier |
|---|---|---|
| B Lymphocytes - G334R | () | 3119 |
| B Lymphocytes - WT | () | 3279 |
| B Lymphocytes - G334R | () | 3518 |
| B Lymphocytes - WT | () | 3550 |
| B Lymphocytes - WT | Coriell Institute | GM11992 |
| B Lymphocytes - R273H | Coriell Institute | GM11994 |
| B Lymphocytes - WT | Coriell Institute | GM18516 |
| B Lymphocytes - Y220H | Coriell Institute | GM18519 |
| B Lymphocytes - P47S | Coriell Institute | GM18523 |
| B Lymphocytes - WT | Coriell Institute | GM18870 |
| B Lymphocytes - P47S homozygote | Coriell Institute | GM18871 |
| B Lymphocytes - E11Q | Coriell Institute | GM18954 |
| B Lymphocytes - P47S | Coriell Institute | GM19092 |
| B Lymphocytes - WT | Coriell Institute | GM19131 |
| B Lymphocytes - P47S | Coriell Institute | GM19143 |
| B Lymphocytes - P47S | Coriell Institute | GM19393 |
| B Lymphocytes - WT | Coriell Institute | GM19394 |
| B Lymphocytes - P47S | Coriell Institute | GM19982 |
| B Lymphocytes - WT | Coriell Institute | GM19984 |
| B Lymphocytes - G360A | Coriell Institute | GM20812 |
| B Lymphocytes - WT | Coriell Institute | GM20814 |
| B Lymphocytes - R110H | Coriell Institute | HG01863 |
| B Lymphocytes - R110H | Coriell Institute | HG02023 |
| B Lymphocytes - R110H | Coriell Institute | HG02085 |
| B Lymphocytes - Y107H | Coriell Institute | HG02634 |
| B Lymphocytes - WT | Coriell Institute | HG02675 |
| B Lymphocytes - P47S | Coriell Institute | HG03297 |
| B Lymphocytes - WT | Coriell Institute | HG03300 |
| B Lymphocytes - WT | Coriell Institute | HG03753 |
| B Lymphocytes - R175C | Coriell Institute | HG03755 |

NOTE:
All p53 SNPs are heterozygous unless otherwise noted.

Immunoblotting. For western blot analyses, 10-50 g of protein was resolved over SDS-PAGE gels using 10% NuPAGE Bis-Tris precast gels (Life Technologies) and were then transferred onto PVDF membranes (pore size: 0.45 mm; Millipore Sigma). Membranes were blocked for 1 hr in 5% bovine serum albumin or 5% non-fat dry milk. Membranes were then incubated overnight in primary antibody at 4 °C while shaking. Rabbit or mouse secondary antibodies conjugated to horseradish peroxidase were used (Jackson ImmunoResearch), followed by a 5-min treatment with ECL. Protein levels were detected using autoradiography.

Antibodies. The following antibodies were used: anti-CEACAM1 (Invitrogen), anti-GAPDH (14C10; Cell Signaling Technology), anti-Histone H3 (acetyl K27; Abcam), anti-Histone H3 (acetyl K27; Abcam), anti-Hsc70 (Proteintech), anti-Ki67 (D3B5; Cell Signaling Technology), anti-LCN15 (Invitrogen), anti-Mdm2 (Cell Signaling Technology), anti-Mdm2 (Ab-1; Sigma-Aldrich), anti-Mdm2 (Ab-2; Sigma-Aldrich), Mouse monoclonal IgG1 isotype control (G3A1; Cell Signaling Technology), anti-NF-κB p65 (D14E12; Cell Signaling Technology), anti-NF-κB p65 (phospho-Ser276; GeneTex), anti-p21 (12D1; Cell Signaling Technology), anti-p53 (1C12; Cell Signaling Technology), anti-p53 (7F5; Cell Signaling Technology), anti-p53 (CM5; Cell Signaling Technology), anti-p53 (DO-1; Santa Cruz Biotechnology), anti-p53 (FL-393; Bioss), anti-p53 (mutant, pAb240; Santa Cruz Biotechnology), anti-PADI4 (GeneTex), anti-PUMA (Cell Signaling Technology), anti-RRAD (LSBio), and anti-TNF-α (GeneTex).

Immunoprecipitation. Tissues were homogenized using the Wheaton Overhead Stirrer in Lysis Buffer (50 mM Tris-HCl, pH 8; 150 mM NaCl, 5 mM EDTA, 0.5% IGEPAL CA-630) with freshly supplemented protease inhibitors at 4 °C. Total cellular homogenates were rotated at 4 °C for 30 min, and spun at 11,000×g for 30 min at 4 °C. Protein extracts (3 mg per reaction) were incubated with the anti-Hsc70 antibody (Proteintech) or control IgG overnight at 4 °C. The Hsc70-immunocomplexes were captured using the Recombinant Protein G Agarose (Thermo Fisher Scientific) at 4 °C for 2 h. The resins were washed three times using the Lysis Buffer. After adding an equal volume of 2× Laemmli Sample Buffer (BioRad) supplemented with 5% β-Mercaptoethanol (BioRad) to each reaction, the samples were heated for 10 min at 100 °C. The Hsc70-associated proteins were size fractionated on Novex 4-20% Tris-Glycine Mini Protein Gels (Thermo Fisher Scientific) at room temperature and subsequently transferred overnight onto Immuno-Blot PVDF membranes (BioRad) at 4 °C. The membranes were blocked with 3% Blotting-Grade Blocker (BioRad) in 1×PBST for 30 min at room temperature and incubated with the anti-p53 antibody (Cell Signaling Technology) overnight at 4 °C. After washing the blots in 1×PBST, the membranes were incubated with Peroxidase AffiniPure F(ab′)₂ Fragment Donkey Anti-Mouse IgG (H+L) (Jackson ImmunoResearch) at 1:10,000 dilution for 2 h at room temperature. Membrane-immobilized protein detection used ECL Western Blotting Detection Reagents (Cytiva RPN2106; MilliporeSigma).

Immunohistochemistry. Tissues were harvested and fixed in formalin overnight at 4 °C, followed by a wash with 1×PBS and then placed in 70% ethanol prior to paraffin embedding. The Wistar Institute Histotechnology Facility performed the tissue embedding and sectioning. Paraffin embedded tissue sections were deparaffinized in xylene and rehydrated in ethanol (100%-95%-85%-75%) followed by distilled water. Samples underwent antigen retrieval by steaming slides in 10 mM Citrate Buffer (pH 6). Endogenous peroxidase activity was quenched with 3% hydrogen peroxide and slides were incubated in blocking buffer for 1 hr. The slides were incubated with primary antibody overnight at 4 °C. Slides were washed with PBS and incubated with HRP-conjugated secondary antibody for 30 min. Antibody complexes were detected using DAB chromogen. Light counterstaining was done with hematoxylin. Slides were imaged on a Nikon 80i upright microscope and at least four fields were taken per section.

Immunofluorescence and proximity ligation assay. Cells were grown on eight-well chamber slides (Lab-Tek), and were treated with 5 μM CDDP for 24 hr. Cells were fixed in 4% paraformaldehyde (Electron Microscopy Sciences) for 10 min, followed by 3 washes in 1×PBS and permeabilization with 0.25% Triton X-100 (Millipore Sigma) for 5 min. For immunofluorescence staining, cells were washed 3× with 1×PBS prior to blocking for 1 hr at room temperature in a blocking buffer consisting of 4% normal goat serum (Jackson Immunoresearch) and 1% bovine serum albumin in PBS. Cells were incubated overnight at 4 °C with primary antibodies diluted in blocking buffer. Cells were washed 3× with 1×PBS and incubated with the following secondary antibodies at room temperature for 1 hr: Alexa Fluor 488 AffiniPure Goat anti-Mouse IgG (Jackson ImmunoResearch) and Alexa Fluor 594 AffiniPure Goat anti-Rabbit IgG (Jackson ImmunoResearch). Cells were mounted with media containing DAPI and images were captured using a Nikon TiE automated inverted microscope. Protein-protein interactions were assessed using the PLA Duolink in situ starter kit (Sigma-Aldrich) following the manufacturer's protocol. Images were captured using a Leica TSC SP5 confocal microscope.

BMH cross-linking. Human LCL cell lines were cultured in DMEM-high glucose supplemented with 10% FBS and 1× penicillin/streptomycin prior to beginning the assay. Cells were transferred to DMEM-high glucose without glutamine, methionine, and cystine, supplemented with freshly added 1% FBS, 1× penicillin/streptomycin, 0.1 mM L-Methionine, 0.5 mM L-Glutamine, and 0.033 mM-0.1 mM L-cystine for up to 24 h. Where appropriate, cells were treated with 1 g/mL arsenic trioxide (ATO; Sigma-Aldrich) overnight. Cells were harvested and cell pellets were resuspended in ice-cold 1×DPBS with 0.5% IGEPAL CA-630 containing freshly added protease inhibitors (1 mM PMSF, 10 g/mL Aprotinin, and 10 g/mL Leupeptin). Samples were lysed by sonication. Lysates were treated with 1 mM BMH and incubated at 30 °C for 30 min. Reactions were quenched with 2× Laemmli sample buffer with freshly added 10% 2-mercaptoethanol and then analyzed by immunoblotting.

In vitro invasion assays. Matrigel invasion assays for MEFs and HCT116 cells were carried out with 24-well BioCoat Matrigel Invasion Chambers with 8.0 μm PET membrane (Corning). In brief, chambers were rehydrated with serum free media and incubated for 2 h at 37 °C and then inserted into a 24-well filled with 750 μL media supplemented with 10% FBS. Approximately 20,000-50,000 cells were seeded in the upper chamber in 500 μL media supplemented with 1% FBS. Cells were allowed to adhere to the Matrigel prior to treating with 20 ng/mL TNF-α, and were then incubated at 37 °C. After 24-30 h, cells that had not migrated into the Matrigel were removed from the upper surface of the membrane with a cotton swab. Inserts were fixed with 100% methanol, stained with 0.5% crystal violet, and imaged for analysis using a Nikon TE2000 Inverted Microscope. Number of invaded cells per field (n=5 random fields of view per insert) were quantified with ImageJ (1.53j, NIH) and expressed as mean±SD. Data are representative of two biological replicates.

RNA isolation and quantitative RT-PCR. Cells were lysed using QIAshredder columns (Qiagen). Total RNA was extracted using the RNeasy Mini kit (Qiagen) and on-column DNase digestions, according to the manufacturer's protocol. Equal amounts of total RNA were then used to generate cDNA using the High-Capacity Reverse Transcription kit (Applied Biosystems) according to the manufacturer's protocol. The resulting cDNA was used with PowerUp SYBR Green Master Mix and the indicated primer sets (see Table S2). Quantitative RT-PCR (Q-RT-PCR) was performed using the QuantStudio 5 Real-Time PCR System (Applied Biosystems). Ct values for the genes of interest were normalized to the invariant control genes. Gene expression data are expressed as relative quantity and normalized to untreated- or vehicle-treated WT controls. All experiments were replicated in at least three independent experiments with three technical replicates in each experiment unless otherwise stated.

TABLE 3
Oligonucleotides for Real-time and ChIP quantitative PCR

| | | |
|---|---|---|
| Target | Forward primer | Reverse Primer |

Primers for Real-time qPCR

| | | |
|---|---|---|
| Human IL-6 | AAATTCGGTACATCCTCGACGGCA | AGTGCCTCTTTGCTGCTTTCACAC |
| | (SEQ ID NO: 5) | (SEQ ID NO: 6) |
| Human IL-8 | ATGACTTCCAAGCTGGCCGT | TCCTTGGCAAAACTGCACCT |
| | (SEQ ID NO: 7) | (SEQ ID NO: 8) |
| Human Fos | CCTACCCGCCACGATGATGT | CTGCGGGTGAGTGGTAGTAA |
| | (SEQ ID NO: 9) | (SEQ ID NO: 10) |
| Human EGFR | ACCTGACCGCAGAGTCTTTT | GAGTGGTTTGGCTGGGGTAA |
| | (SEQ ID NO: 11) | (SEQ ID NO: 12) |

Primers for ChIP qPCR

| | | |
|---|---|---|
| Human IL-6 | AGACATGCCAAAGTGCTGAGT | GAGGCTAGCGCTAAGAAGCA |
| | (SEQ ID NO: 13) | (SEQ ID NO: 14) |
| Human IL-8 | TGGGCCATCAGTTGCAAA | ACTTATGCACCCTCATCTTTTCAT |
| | (SEQ ID NO: 15) | (SEQ ID NO: 16) |
| Human p21 | AGCAGGCTGTGGCTCTGATT | CAAAATAGCCACCAGCCTCTTCT |
| | (SEQ ID NO: 17) | (SEQ ID NO: 18) |
| Human desert | CATCCCTGGACTGATTGTCA | GGTTGGCCAGGTACATGTTT |
| | (SEQ ID NO: 19) | (SEQ ID NO: 20) |

Nuclear fractionation and sequential salt extraction. Cells were harvested and washed twice with ice cold PBS prior to resuspending in Buffer A (25 mM HEPES pH 7.6, 25 mM KCl, 5 mM MgCl2, 0.05 mM EDTA, 0.1% NP-40, 10% glycerol) supplemented with protease inhibitors. Cells were rotated at 4 °C for 10 min and then centrifuged at 6000×g for 5 min at 4 °C to isolate nuclei. Each nuclear pellet was resuspended and homogenized in mRIPA (100 mM Tris pH 8.0, 2% NP-40, 0.5% sodium deoxycholate), incubated on ice for 3 min, and then centrifuged at 6500×g for 3 min at 4 °C to isolate the chromatin pellet. The supernatant (soluble nuclear fraction) was transferred to a clean tube before proceeding with sequential salt extraction of the chromatin pellet. Each chromatin pellet was resuspended and homogenized in mRIPA with sequentially increasing concentrations of NaCl (100 mM-500 mM), incubated on ice for 3 min, and centrifuged at 6500×g for 3 min at 4 °C to extract chromatin bound proteins. NuPAGE Sample Buffer (supplemented with 5% (3-mercaptoethanol) was added to each fraction. Samples were loaded into an SDS-PAGE gel for western blot analysis.

Chromatin immunoprecipitation-quantitative PCR. LCLs (2.0×10⁷) were treated with vehicle control or 0.5 ng/mL TNF-α for the indicated timepoints and then crosslinked with 1% formaldehyde for 10 minutes at room temperature and quenched with 2.5 M glycine for 5 minutes. Crosslinked cells were washed twice in PBS, collected by centrifugation, lysed in 10 ml swelling buffer (10 mM Tris, pH 7.5, 2 mM MgCl2, 3 mM CaCl₂)), and placed on ice for 10 minutes. Chromatin fragmentation was performed with a Bioruptor sonication bath (Diagenode) at 4 °C with the following settings: 25 cycles of high amplitude for 30 seconds, with a 30 second pause in between each sonication. Chromatin was then cleared with centrifugation at maximum speed for 30 minutes at 4 °C, 5% saved for input, and immunoprecipitated with the appropriate antibodies overnight at 4 °C using magnetic Dynabeads Protein G beads (ThermoFisher). Beads were subsequently subjected to the following washes: 2×5 min in ChIP-buffer (50 mM HEPES, pH 7.5, 155 mM NaCl, 1.1% Triton X-100, 0.11% NaDeoxycholate, 1 mM EDTA), 5 min in ChIP-buffer with an additional 500 nM NaCl, 5 min in TE buffer (10 mM Tris-HCl pH 8.0, 1 mM EDTA). The crosslink was reversed overnight at 65 °C with elution buffer (50 mM Tris-HCl pH 8.0, 10 mM EDTA, 1% SDS) before treatment with 0.33 mg/ml RNase A (ThermoFisher) and 0.5 mg/ml Proteinase K (Thermo) at 37 °C for 2 h. DNA was isolated with phenol/chloroform followed by overnight ethanol precipitation at −20 °C. Q-RT-PCR was used to determine the enrichment of immunoprecipitated DNA relative to the input DNA using gene-specific primer sets to the specified regions. Primers directed to a gene-desert were used as a negative control. ChTP-Q-RT-PCR experiments were performed in two independent biological replicates.

Bulk RNA-Seq Analysis. RNA-Seq data from the first experiment studied the effect of Nutlin in LCLs from individuals with P47S and Y107H was aligned using STAR (Dobin A et al Bioinformatics. 2013 Jan. 1; 29(1):15-21) against hg19 version of human genome and RSEM (Li B et al. BMC Bioinformatics. 2011 Aug. 4; 12:323) was used for estimation of read counts and FPKM values using Ensemble transcriptome information. DESeq2 package (Love M I et al. Genome Biol. 2014; 15(12):550) was used for normalization and differential expression analysis. For the RNA-Seq experiment to identify a p53 hypomorph gene signature, the samples in triplicates or duplicates were assayed among three runs with the same two samples (one hypomorph and one wild-type) repeated among the runs. Alignment was done using bowtie2 (Langmead B et al. Nat Methods. 2012 Mar. 4; 9(4):357-9) against hg19 version of human genome and Ensemble transcriptome information followed by RSEM to estimate read counts on gene level. Normalization was carried out using DESeq2 (Love M I et al. Genome Biol. 2014; 15(12):550) and batch correction was performed using the two samples that were repeated in each of the 3 runs. Python's sci-kit learn (sklearn v0.24.2) (Pedregosa F et al. J. Mach. Learn. Res. 12, 2825) was used to train the SVM with an RBF kernel with leave-one-out (LOO) cross validation and recursive feature elimination (RFE) to select features based on the training set (Cortes, C., Vapnik, V. Support-vector networks. Mach Learn 20, 273-297 (1995)). The best 2000 genes based on t-test on log 2-scaled normalized counts were used in each iteration of LOO and RFE was applied removing 10% of features with the lowest SVM scores at each step with the model retrained and the left-out group classified to produce a probability classification score. Probability scores were used to calculate accuracy using 0.5 threshold and area under the ROC curve. The optimal number of genes were selected based on highest accuracy and AUC of the training set. RFE iteration with 32-gene model showed the highest accuracy and AUC with a total of 143 unique genes across all steps of LOO iterations. The SVM was re-trained on the training set with these 143 genes and applied on a set of blinded samples from 6 patients. R package ggplot2 v3.3.5 was used for plotting graphs and figures. Qiagen Ingenuity Pathway Analysis (IPA) was used for enrichment analyses of genesets (Kramer A et al. Bioinformatics. 2014 Feb. 15; 30(4):523-30). The RNA-seq data was deposited to the NCBI GEO database, and can be found under accession number GSE209837.

Mouse Models. All mice were of the C57Bl/6J strain. WT and P47S mice were generated previously in the humanized p53 knock-in backbone (Hupki) (Jennis M et al. Genes Dev. 2016 Apr. 15; 30(8):918-30). Y107H Hupki mice were generated by micro-injection of Cas9/gRNA ribonucleoprotein complex along with single-stranded oligo deoxyribonucleotide donor (ssODN) into single-cell embryos of WT-Hupki mice in the Transgenic Facility at the Fox Chase Cancer Center. The gRNA recognition site is aaaacctaccagggcagctaCGG (SEQ ID NO: 21), with the PAM site in upper case. The ssODN has the following sequence: tgaccgtgcaagtcacagacttggctgtcccagaatgcaagaagcccagacggaaaccgtGGGAgccctggtaggttttctggga agggacagaagatgacaggggccaggagggggctggtgc (SEQ ID NO: 22). The single-piece synthetic gRNA and ssODN containing two phosphorothioate bonds at each end were ordered from Integrated DNA Technologies and validated in human K562 cells prior to microinjection for efficient introduction of the Y107H mutation. Two independent founders with heterozygous T to C mutations were bred to homozygosity and crossed to C57Bl/6 background for five generations. The entire p53 coding region was amplified and sequenced by the Genomics Facility at the Wistar Institute.

Preclinical analyses. All animal studies were completed in accordance with the recommendations in the Guide for the Care and Use of Laboratory Animals of the NIH. All protocols were approved by The Wistar Institute Institutional Animal Care and Use Committee (IACUC). Mice were housed in plastic cages with ad libitum diet and maintained with a 12 hr dark/12 hr light cycle at 22 °C. For in vivo induced chronic colitis assays, 8- to 10-week-old male and female mice were administered 2% dextran sulfate sodium (DSS; Alfa Aesar) in three 4-day cycles separated by 17-day intervals. Mice were monitored three times per week for overall weight, stool consistency, and gross bleeding. Inflammatory score assessment was conducted in blinded manner by a pathologist (D. Garlick, StageBio).

Statistical analyses. Unless otherwise specified, all experiments were performed in three independent biological replicates and data were analyzed using the two-tailed unpaired t-test. All in vitro data are reported as the mean±SD unless stated otherwise. For data containing more than 2 experimental groups, one-way ANOVA with Tukey's post-test analysis was performed. All statistical analyses were performed in GraphPad Prism (v9.0).

### Example 2

### Results

Altered p53 Target Gene Expression in Lymphoblastoid Cell Lines from P47S and Y107H Individuals

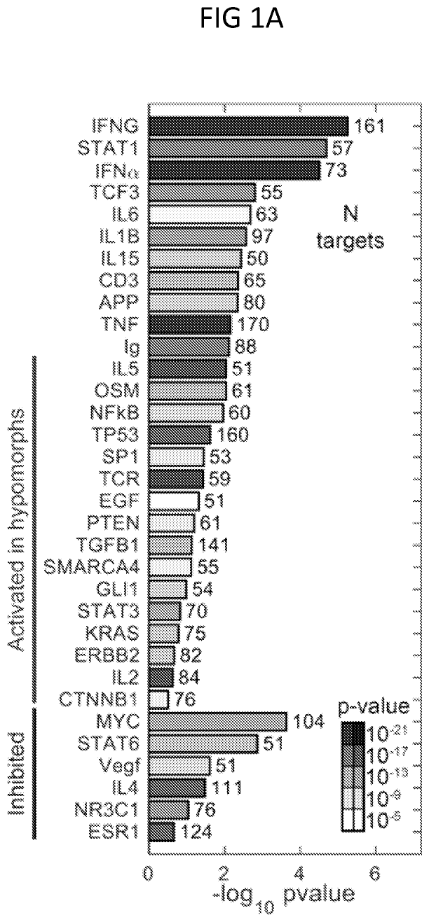

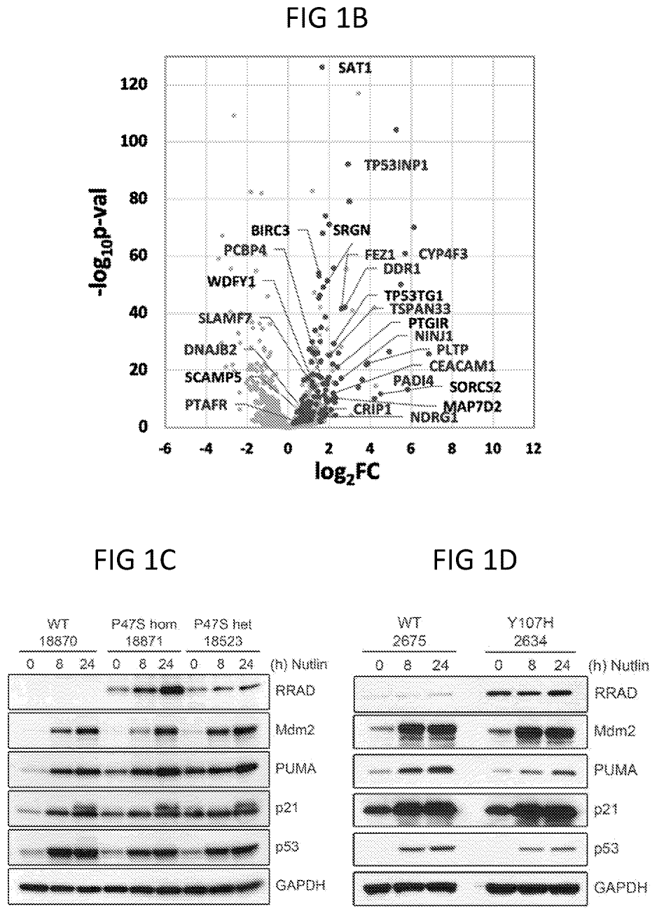

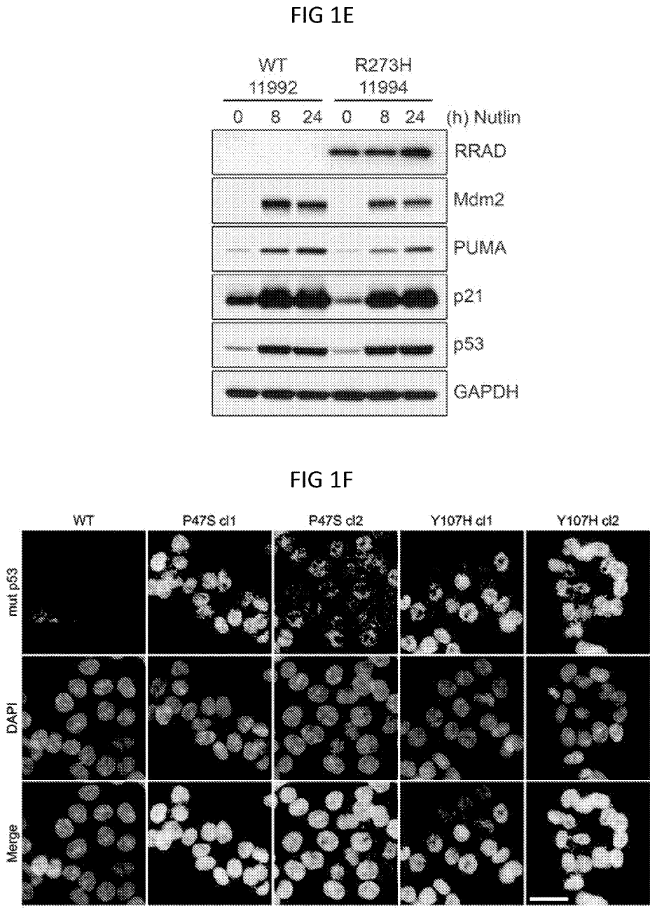

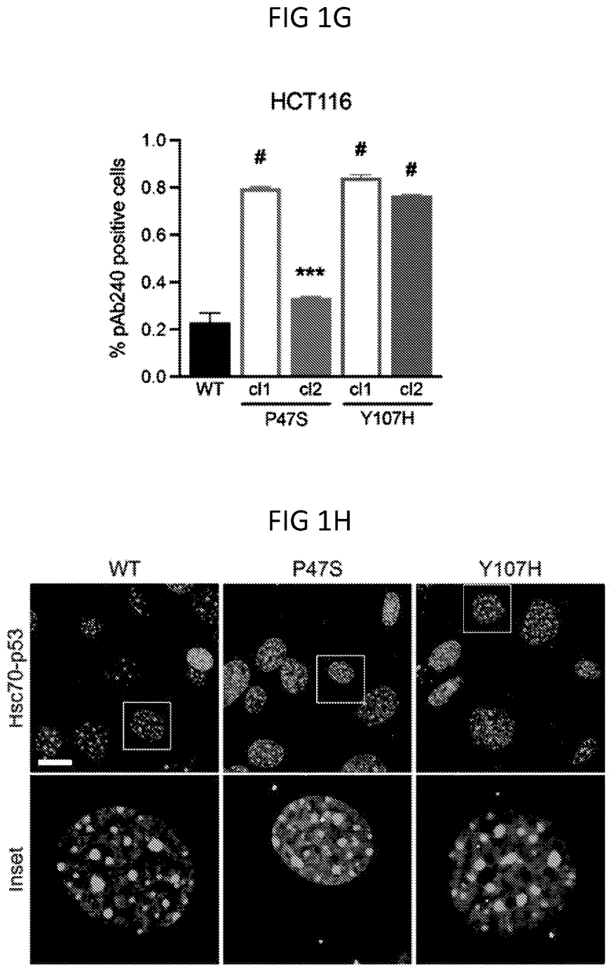

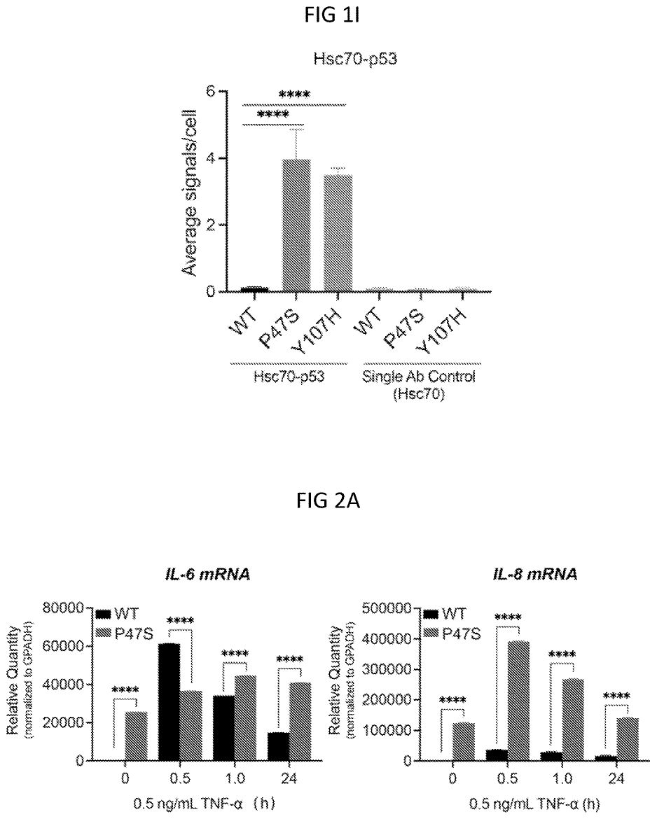

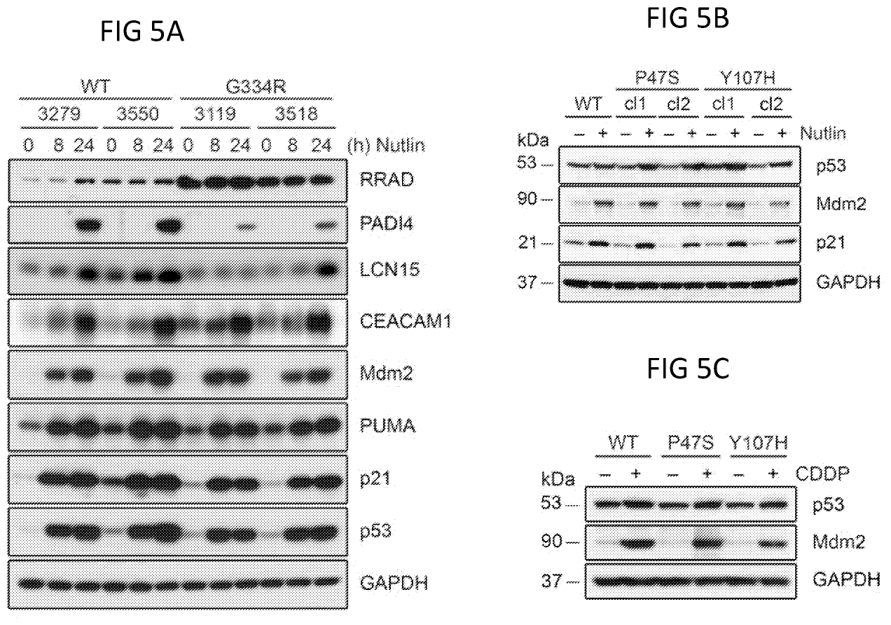

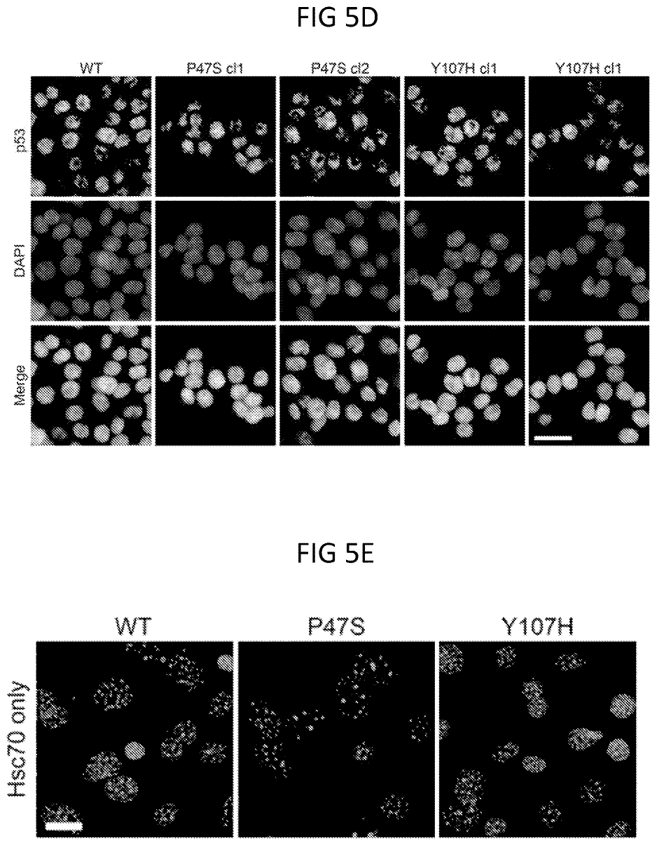

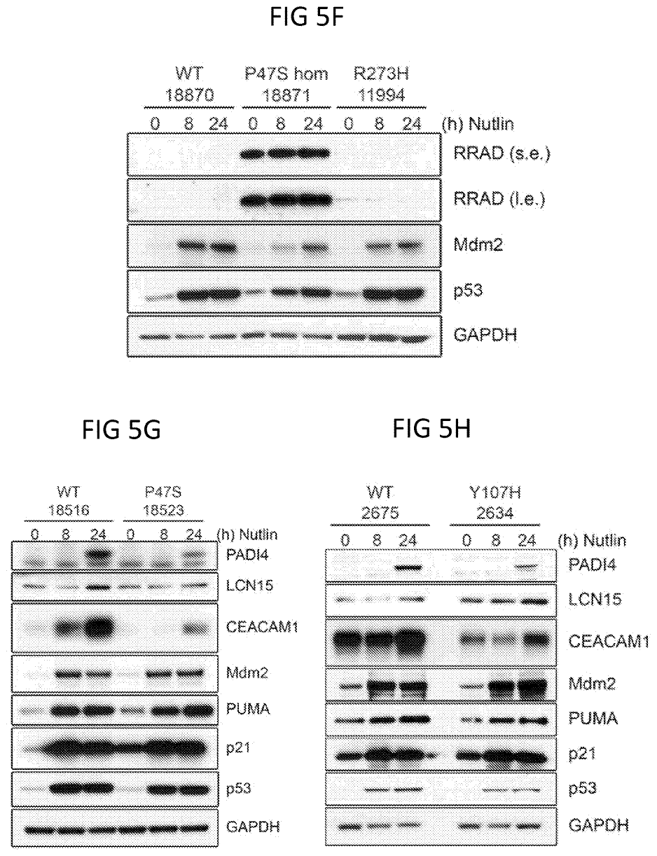

We sought to assess the p53 transcriptional response in lymphoblastoid cell lines (LCLs) isolated from individuals containing two different germline genetic variants of TP53: P47S (Pro47Ser, rs1800371) and Y107H (Tyr107His, rs368771578). Toward this goal we conducted RNA Sequencing (RNA Seq) and western blot analysis following Nutlin-3a treatment in P47S and Y107H LCL lines, compared to age- and sex-matched WT controls from the same region of Africa (Y107H) or the same family (P47S). Because these hypomorphic variants are located in distinct functional domains of p53 (P47S in transactivation domain 2, and Y107H in the DNA binding domain of p53) we were surprised to find three things in common between them. First, we found evidence for an enhanced NF-κB gene signature in hypomorph cells relative to controls, even under unstressed conditions (FIG. 1A). Second, we identified a small subset of p53 target genes that showed impaired transactivation in both hypomorph lines following Nutlin-3a treatment compared to controls (FIG. 1B); this occurred despite equal induction of p53 and the p53-induced targets MDM2, p21/CDKN1A and PUMA in P47S, Y107H, and WT cells (FIG. 1C, FIG. 1D). Third, we discovered one p53 target, RRAD, that was markedly increased at the protein level in P47S and Y107H cells, both at baseline and following Nutlin-3a treatment, compared to controls. RRAD was highest in homozygous P47S LCLs, but also was increased in P47S and Y107H heterozygous LCLs relative to controls (FIG. 1C, FIG. 1D). RRAD was also higher in LCLs from another heterozygous p53 hypomorph, G334R (23) compared to a WT sibling control (FIG. 5A), and in heterozygous LCLs with a Li Fraumeni mutant, R273H (FIG. 1E). Our analyses indicate that these two hypomorphs are defective in the transactivation of only a small subset of p53 target genes, and thus can be classified as hypomorphic. They also suggest that there might be a correlation between RRAD level and the presence of inactive or hypomorphic p53.

### p53 Hypomorphs Adopt a Mis-Folded, Mutant Conformation

RRAD is regulated by both p53 and NF-κB (24). Mutant forms of p53, including the R273H mutant, bind and enhance NF-κB activity (9). This led us to predict that the P47S and Y107H variants might both misfold into the common, denatured conformation that is characteristic of mutant p53 and specifically recognized by the monoclonal antibody pAb240 (25, 26). We further reasoned that P47S and Y107H LCLs might show increased NF-κB activity. To test the first hypothesis, we used CRISPR-Cas9 knock-in technology to generate two clones each of the P47S and Y107H hypomorphs, in the background of HCT116 cells (FIG. 5B and FIG. 5C). We also used CRISPR/Cas9 to generate the Y107H knock-in mouse using C57Bl/6 embryos; these Y107H mice were backcrossed to a C57Bl/6 background for five generations, and the entire p53 coding region was sequenced to ensure the absence of second-site mutations. Immunofluorescent staining with the pAb240 mutant conformation-specific antibody detected a significantly increased percentage of mutant p53 conformation-positive cells in P47S and Y107H knock-in HCT116 clones compared to the parental WT p53 cell line; there were approximately 20% pAb240 positive cells in WT, and 35-80% pAb-240 positive cells in hypomorph clones (FIG. 1F, FIG. 1G). Staining with the pan-p53 antibody was indistinguishable (FIG. 5D). As an orthogonal approach, we analyzed primary mouse embryonic fibroblasts (MEFs) isolated from mice containing germline versions of WT p53, P47S or Y107H, all generated in the Humanized p53 knock-in background (16). We first confirmed equal induction of p53 and MDM2 in these MEFs following genotoxic stress (FIG. 5C). In these WT, P47S and Y107H MEFs we used proximity ligation analysis (PLA) to assess the interaction of p53 with Hsc70, as this interaction is specific for misfolded, mutant p53 (27, 28). PLA of the Hsc70-p53 interaction in early passage primary MEFs revealed significantly increased association of Hsc70 with P47S and Y107H compared to WT p53 (FIG. 1H, FIG. 5I; see FIG. 5E for the negative control). These data indicate that the P47S and Y107H hypomorphs both have increased propensity to misfold into a mutant conformation. This is similar to what we showed previously for the G334R hypomorph of p53 (23).

We next sought to determine whether the level of RRAD might correlate with the level of misfolded p53 in the cell. First, we noted that there was increased RRAD in homozygous P47S LCLs, compared to heterozygous (FIG. 1C). Second, we found that the level of RRAD was significantly greater in cells containing the P47S variant compared to R273H; the latter is generally believed to be predominantly folded into a WT conformation (FIG. 5F). Next, we sought to confirm our RNA Seq findings that a subset of p53 target genes were transactivated less in P47S and Y107H LCLs; western blot analysis confirmed that the p53 target proteins PADI4 and CEACAM-1 exhibit decreased induction in P47S and Y107H LCLs relative to matched WT controls (FIG. 5G, FIG. 5H). In contrast the p53 targets LCN15, MDM2, PUMA, and p21/CDKN1A were induced to equivalent levels in hypomorph and WT LCLs (FIG. 5G, FIG. 5H). The combined data indicate that increased RRAD levels and decreased ability to transactivate a subset of target genes is shared between the P47S and Y107H hypomorphic variants of p53.

### P47S and Y107H Cells Exhibit Elevated NF-κB Signaling

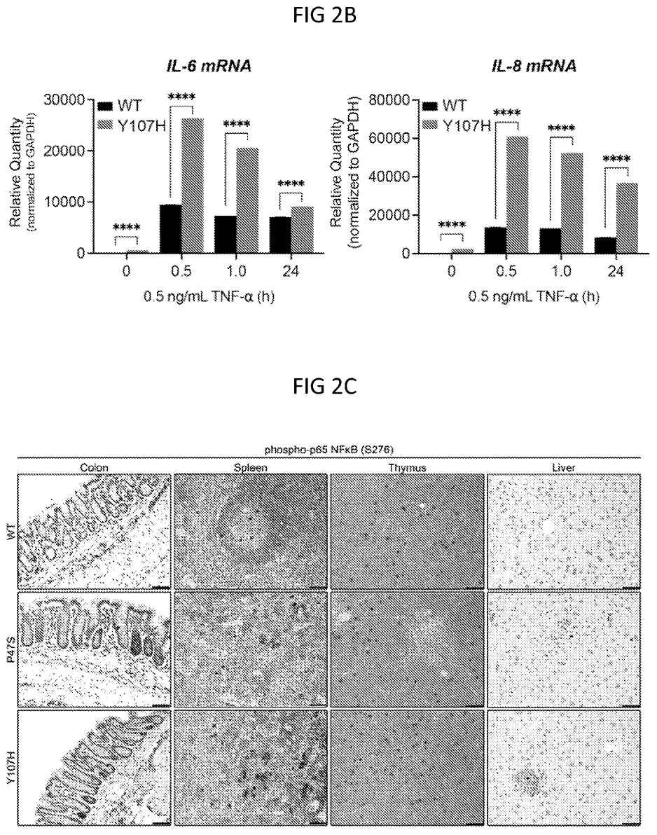

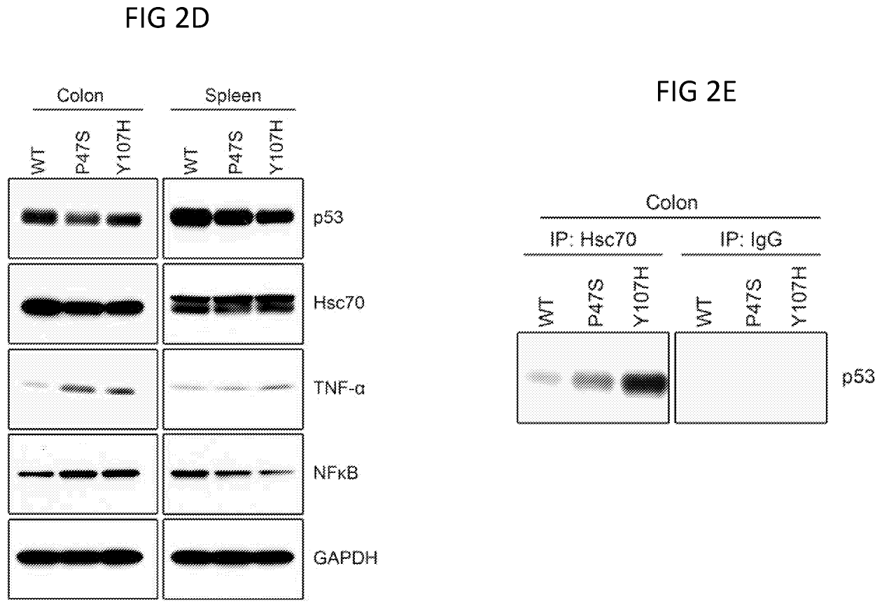

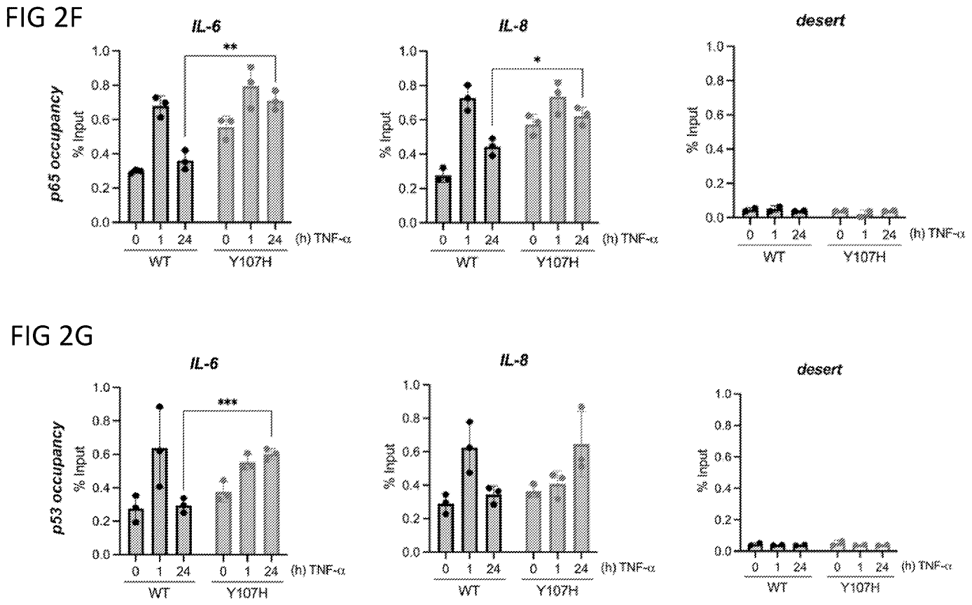

Tumor-derived mutant forms of p53 can bind and stabilize the p65 subunit of NF-κB on chromatin, leading to increased steady state level of NF-κB target genes in tumors with mutant p53 (9, 29, 30). We hypothesized that the increased mutant conformation of p53 in P47S and Y107H cells might lead to increased NF-κB activity. To address this, we treated LCLs heterozygous for P47S and Y107H with TNF-α and analyzed the induction of IL-6 and IL-8, two well-known NF-κB target genes by quantitative real time polymerase chain reaction (q-RT-PCR). In both cases we compared this induction to matched WT LCL lines. This analysis revealed that both P47S and Y107H LCLs exhibit hyperactive NF-κB signaling compared to matched WT controls, both at baseline and in response to stimulation with TNF-α (FIG. 2A, FIG. 2B). Expression of NF-κB target genes remained elevated for 24 hours in p53 hypomorph cells relative to WT controls, similar to the previously reported “gain of function” activity of mutant p53 (9). In contrast, we failed to see increased transcriptional response to another stimulus, epidermal growth factor (EGF), in p53 hypomorph cells relative to control (FIG. 5A, FIG. 5B).

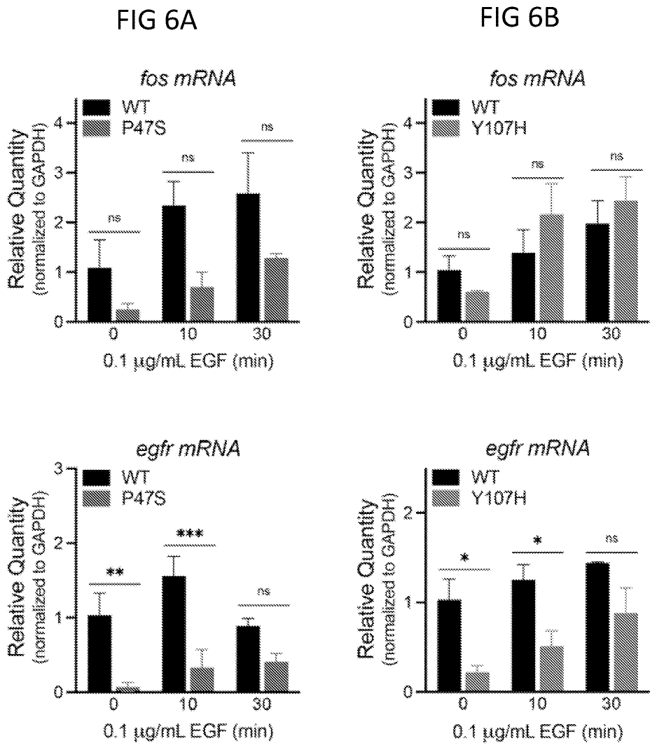

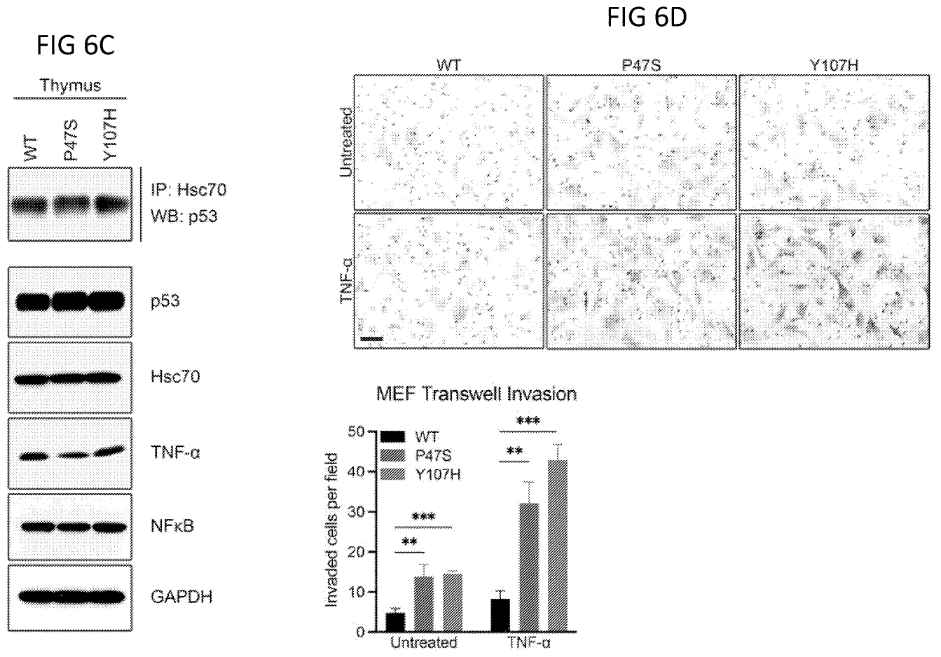

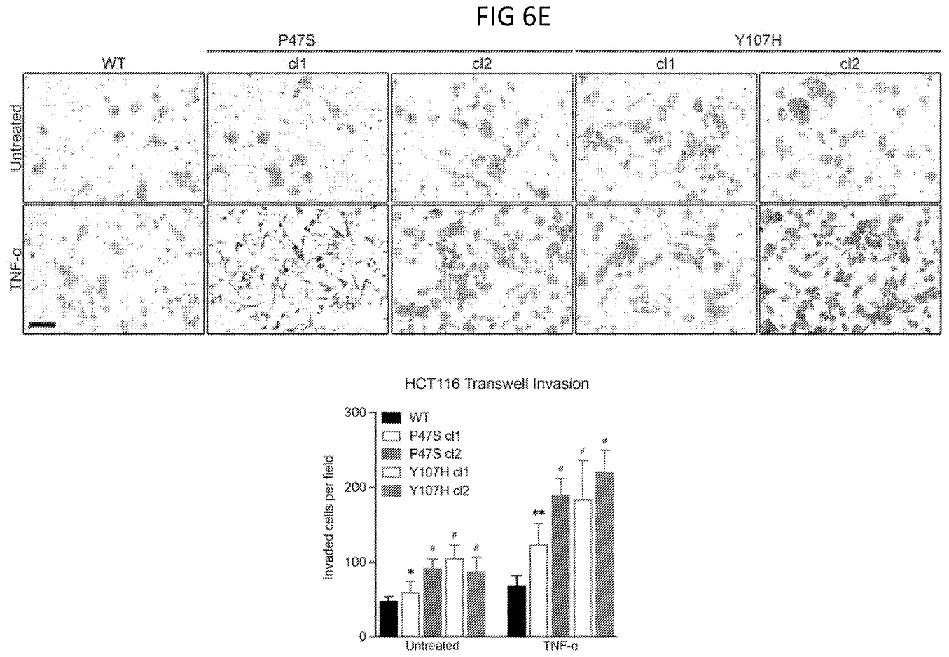

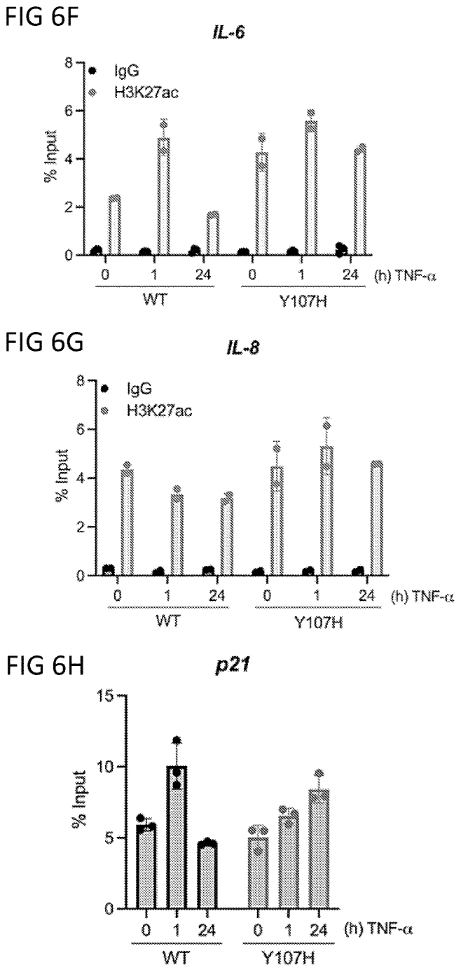

To corroborate our findings, we next sought to probe NF-κB signaling in tissues from p53 hypomorph mice. We used immunohistochemistry to analyze the level of phospho-p65 (S276) in the colon, spleen, thymus and liver of mice containing the P47S and Y107H variants, compared to WT p53. We discovered markedly elevated phospho-p65 staining in the colons and spleens of P47S and Y107H hypomorph mice relative to WT controls (FIG. 2C). In contrast, there were no differences in phospho-p65 staining in the thymus or livers of these mice compared to controls, suggesting that these findings were cell type-specific. Additionally, western blot analysis confirmed equal steady state level of p65 and p53 in these mice (FIG. 2D). We also noted evidence for increased TNF-α in the colon and spleens of these mice, which was consistent with increased NF-κB activity (FIG. 2D). We next monitored the Hsc70-p53 interaction in the colon and thymus, and found increased Hsc70 association with P47S and Y107H in the colon, consistent with the premise that increased misfolded p53 tracks with increased NF-κB activity (FIG. 2E). We saw equivalent Hsc70-p53 interaction in the thymus of WT, P47S and Y107H mice, and no difference in TNF-α levels in the thymus of these mice (FIG. 6C). These data indicate that increased NF-κB activation occurs in a subset of tissues from hypomorph mice, and is correlated with the level of Hsc70-p53 interaction.

One of the most consistent phenotypes conferred by mutant p53 is increased invasiveness (14). We therefore next explored the propensity for invasion in MEFs and HCT116 cells containing P47S, Y107H or WT p53. To do this, we performed invasion assays with cells that were untreated, or that were treated with TNF-α to stimulate migration. We found that MEFs from P47S and Y107H mice exhibited significantly increased migration and invasion compared to WT controls, both when untreated and in the presence of TNF-α (FIG. 6D). Similar results were obtained in P47S and Y107H HCT116 clones, compared to WT (FIG. 6E). In sum, multiple clones of human and mouse cells containing the P47S and Y107H hypomorphs showed increased invasive potential, both at steady state and following stimulation with TNF-α.

### Increased p65 Occupancy on Chromatin in Y107H LCLs

Mutant p53 binds to and prolongs NF-κB occupancy on chromatin at the promoters of NF-κB target genes (9). We therefore next performed chromatin immunoprecipitation (ChIP) for p65 and p53 at the promoters of NF-κB target genes in WT and Y107H hypomorph LCLs. This analysis revealed increased occupancy of p65 at the promoters of IL-6 and IL-8 in Y107H cells, compared to WT, both at steady state and following treatment with TNF-α (FIG. 2F). Notably, we also found evidence for p53 occupancy at the NF-κB sites in these promoters (FIG. 2G). Primers for a promoter desert and IgG controls were clear of signal, and control CDKN1A primers showed comparable signal in WT and Y107H LCLs (FIG. 2F, FIG. 2G; FIG. 6F-FIG. 6H).

### Altered Biochemical Properties of the Y107H Hypomorph can be Reversed by the p53 Refolding Agent Arsenic Trioxide (ATO)

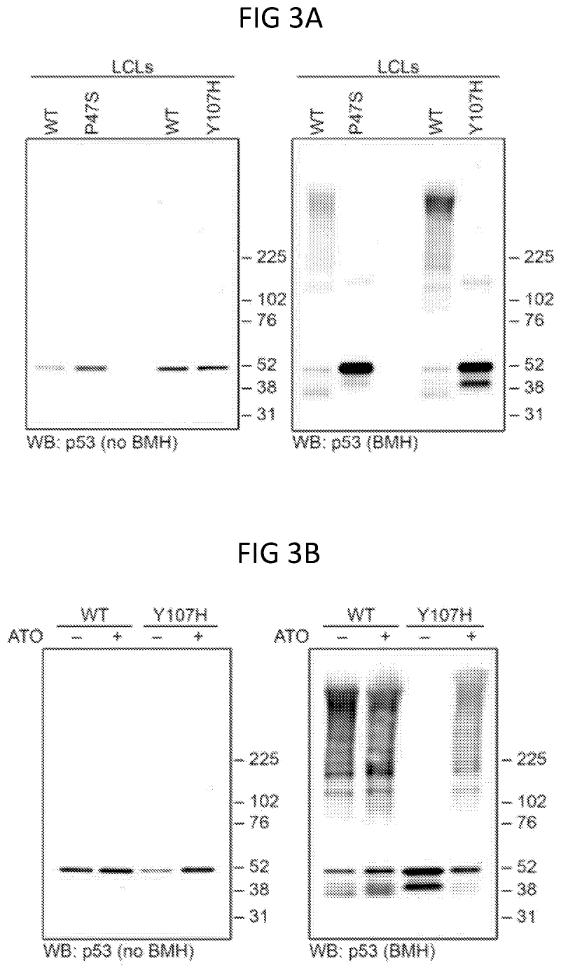

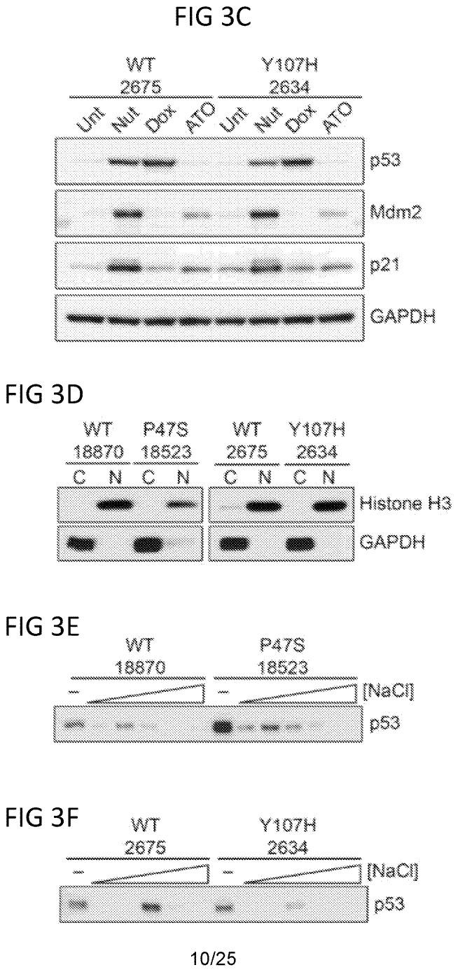

We next sought to analyze the ability of hypomorphic p53 to participate in protein-protein interactions, including the ability to form tetramers and higher-order oligomers. To do this, we employed the cross-linking agent bismaleimidohexane (BMH), which we have previously shown can detect species consistent with p53 tetramers and octamers, along with higher-order protein complexes (18). As expected, untreated LCLs from P47S and Y107H showed similar steady state levels of p53 compared to matched WT cells (FIG. 3A). Following BMH cross-linking, there were marked differences in the pattern of crosslinked species in hypomorph cells compared to WT p53: the majority of WT p53 existed in higher molecular weight complexes, consistent with p53 tetramers and octamers, while the P47S and Y107H proteins appear to exist predominantly as monomers, along with minor species consistent with the size of p53 dimers (FIG. 3A). Interestingly, the higher molecular weight complexes could be restored for the Y107H hypomorph by incubation with arsenic trioxide (ATO), which is reported to refold mutant p53 into a WT conformation (31). ATO had no effect on WT p53 protein complex formation (FIG. 3B), nor was this concentration of ATO sufficient to stabilize p53 (FIG. 3C). To explore the ability of hypomorphs to bind to chromatin, we next analyzed WT, P47S and Y107H LCLs for the presence of p53 in nuclear, salt-extracted fractions. We did not see significant differences in the percent of WT, P47S or Y107H p53 in the salt-extractable, presumably chromatin-bound, state (FIG. 3D-FIG. 3F). The combined data support the premise that p53 hypomorphs do not show overt differences in chromatin binding compared to WT p53, but they show reduced propensity to engage in higher-order tetramers and oligomers in unstressed cells.

### Identification of a Gene Expression Signature for Hypomorphic p53

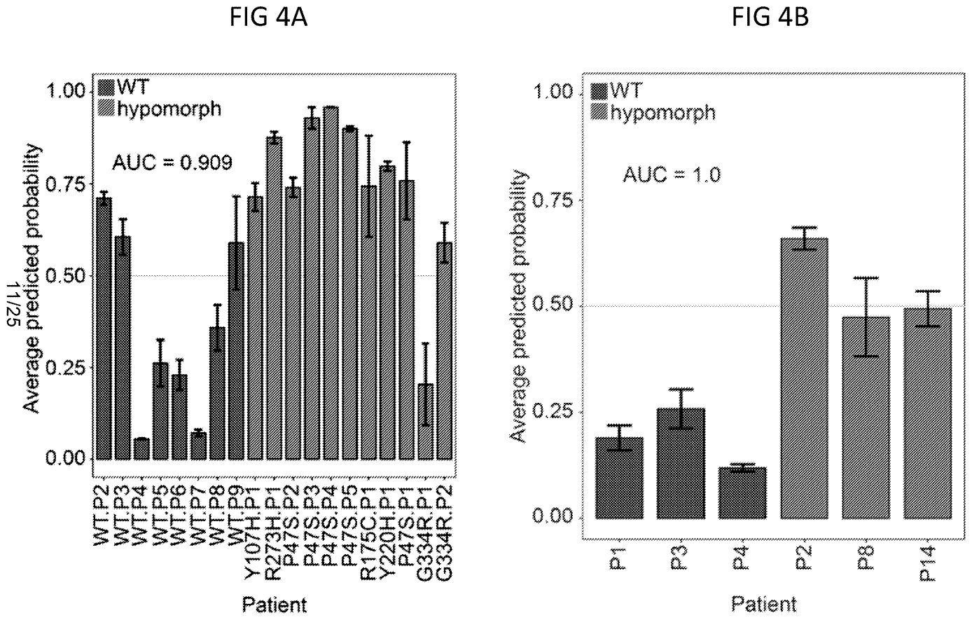

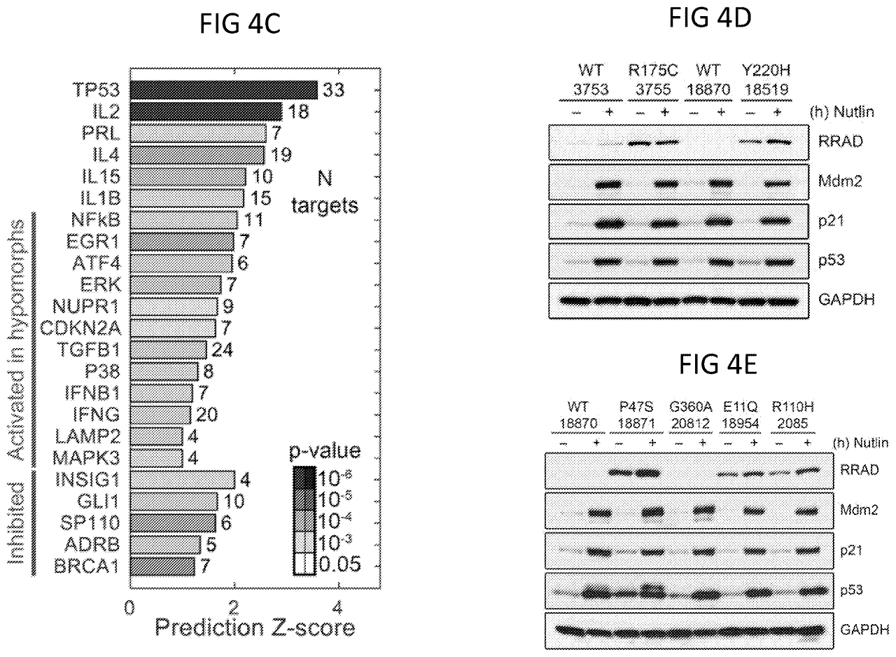

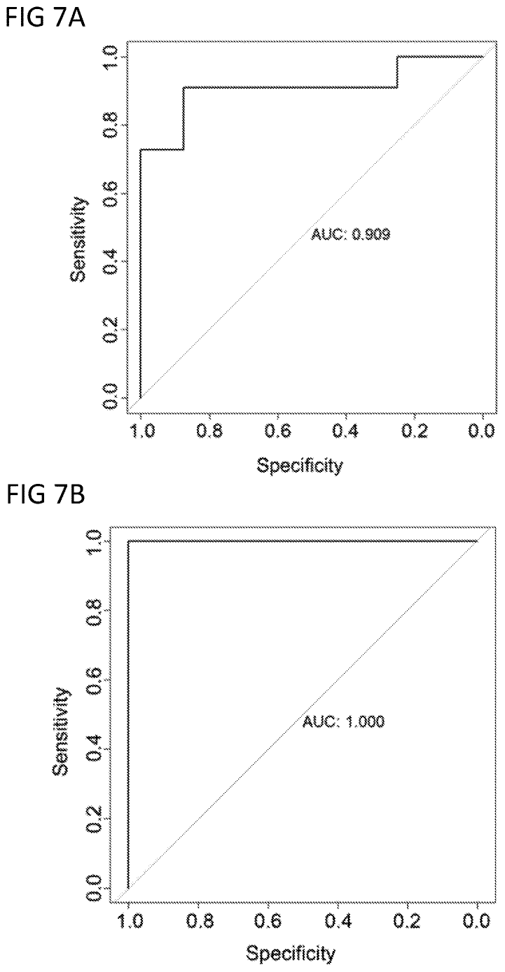

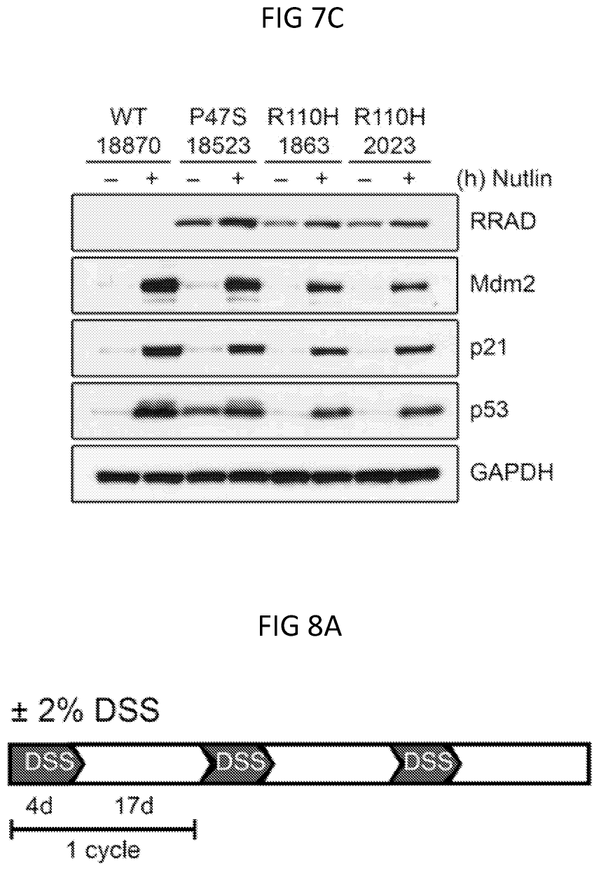

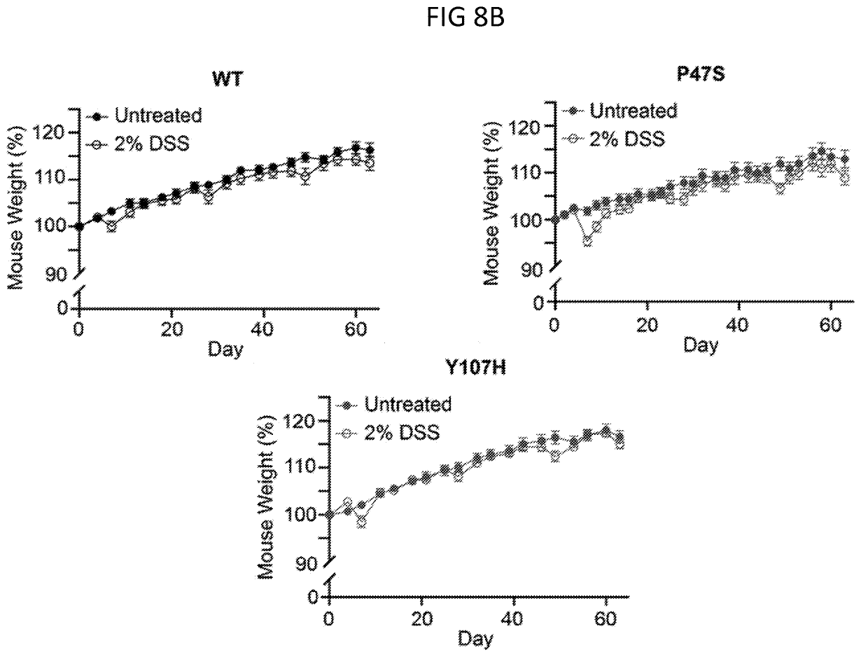

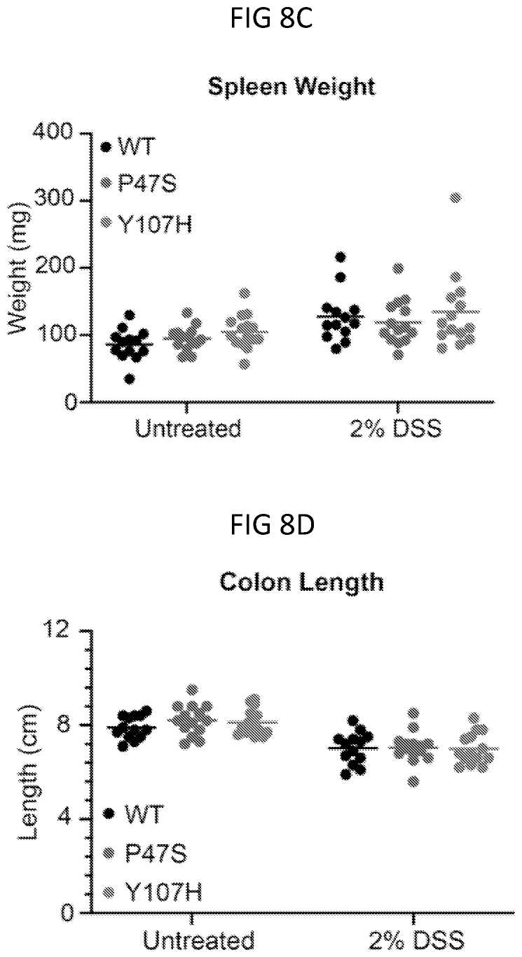

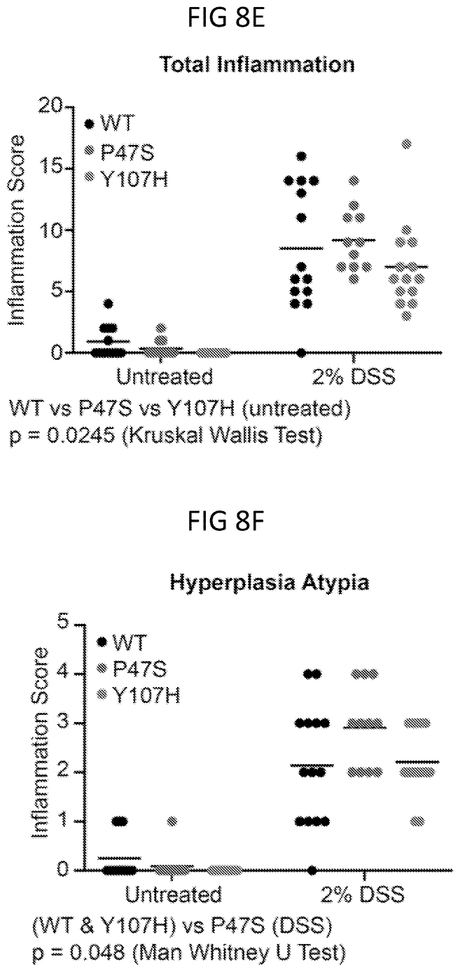

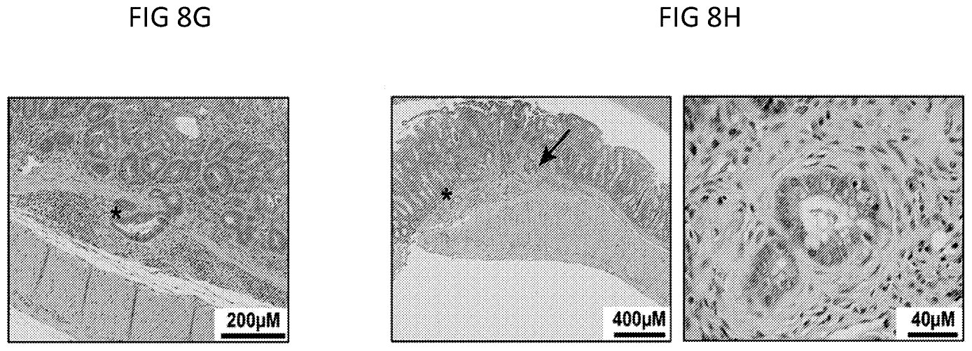

Our finding of increased NF-κB and altered gene expression in unstressed LCLs from P47S and Y107H individuals prompted us to test the possibility that these and other hypomorphs might share a common gene signature. Toward this goal, we compared RNA Seq data from RNA isolated from eighteen samples. This included eight different LCL lines with WT p53 (of varied ethnicity, sex, and age) along with ten different hypomorph cell lines: Y107H; P47S (5 independent lines); R175C; Y220H; G334R and R273H (Li Fraumeni mutant). With the exception of one P47S line, all LCLs were heterozygous. All lines were analyzed in unstressed conditions in biological triplicates. Gene expression data were used to train a support vector machine classifier model that identified a 143-gene signature with the potential to distinguish between WT p53 and a hypomorph (FIG. 4A; FIG. 7A-FIG. 7C). This 143-gene signature was then assessed against a blinded independent test set of six new samples: two were previously unanalyzed WT samples, two were previously unanalyzed hypomorphs (P47S) and two samples were of unknown functional status (G360A and E11Q). This model accurately predicted all four WT and P47S hypomorph variants. It classified E11Q as a hypomorph, and G360A as WT, suggesting that the latter is a benign variant (FIG. 4B, FIG. 7B). Ingenuity pathways analysis (IPA) of these genes indicated that significant regulators in the hypomorph gene signature were, not surprisingly, p53 and NF-κB (FIG. 4C). Importantly, we next made an effort to confirm the accuracy of our gene signature predictions in the validation set by using the steady state level of RRAD as a surrogate for hypomorphic p53; our RRAD western blots confirmed the conclusions of our gene signature prediction, that E11Q is likely a hypomorph, while G360A is likely a benign variant (FIG. 4D, FIG. 4E). Table 4 below shows differential expression for a representative p53 hypomorph sample.

TABLE 4

| EnsembleID | Symbol | fold (hypomorph/benign) |
|---|---|---|
| ENSG00000076650 | GPATCH1 | −1.16208 |
| ENSG00000068097 | HEATR6 | −1.29758 |
| ENSG00000100028 | SNRPD3 | 1.117983 |
| ENSG00000100412 | ACO2 | 1.139409 |
| ENSG00000102265 | TIMP1 | 1.391297 |
| ENSG00000104490 | NCALD | 2.473321 |
| ENSG00000109861 | CTSC | 1.306173 |
| ENSG00000110841 | PPFIBP1 | 1.657327 |
| ENSG00000111729 | CLEC4A | −1.15682 |
| ENSG00000010810 | FYN | 4.112615 |
| ENSG00000119397 | CNTRL | −1.26944 |
| ENSG00000121579 | NAA50 | −1.18703 |
| ENSG00000122642 | FKBP9 | 1.590197 |
| ENSG00000122862 | SRGN | 1.732382 |
| ENSG00000123892 | RAB38 | 2.595734 |
| ENSG00000124766 | SOX4 | 1.378945 |
| ENSG00000125450 | NUP85 | −1.25738 |
| ENSG00000125657 | TNFSF9 | 1.449851 |
| ENSG00000126262 | FFAR2 | 2.466249 |
| ENSG00000126767 | ELK1 | 1.30894 |
| ENSG00000126787 | DLGAP5 | −1.28703 |
| ENSG00000127666 | TICAM1 | 1.210617 |
| ENSG00000100227 | POLDIP3 | −1.09763 |
| ENSG00000130303 | BST2 | 1.180186 |
| ENSG00000130513 | GDF15 | 1.848008 |
| ENSG00000089280 | FUS | −1.25467 |
| ENSG00000134590 | FAM127A | 1.377692 |
| ENSG00000134644 | PUM1 | 1.120217 |
| ENSG00000135597 | REPS1 | 1.272168 |
| ENSG00000135838 | NPL | 2.307756 |
| ENSG00000111913 | FAM65B | −1.57398 |
| ENSG00000083937 | CHMP2B | 1.155795 |
| ENSG00000109220 | CHIC2 | 1.307616 |
| ENSG00000080819 | CPOX | 1.542564 |
| ENSG00000115539 | PDCL3 | −1.41527 |
| ENSG00000059728 | MXD1 | 1.288165 |
| ENSG00000075785 | RAB7A | 1.205898 |
| ENSG00000038427 | VCAN | 2.323797 |
| ENSG00000120727 | PAIP2 | 1.283951 |
| ENSG00000041802 | LSG1 | −1.13181 |
| ENSG00000119048 | UBE2B | 1.180818 |
| ENSG00000029534 | ANK1 | 2.412284 |
| ENSG00000075239 | ACAT1 | 1.110462 |
| ENSG00000139192 | TAPBPL | −2.06009 |
| ENSG00000110934 | BIN2 | −1.56444 |
| ENSG00000140263 | SORD | 1.408954 |
| ENSG00000051180 | RAD51 | −1.33778 |
| ENSG00000140374 | ETFA | 1.215801 |
| ENSG00000143195 | ILDR2 | 3.410116 |
| ENSG00000144655 | CSRNP1 | 1.507565 |
| ENSG00000145779 | TNFAIP8 | −1.19191 |
| ENSG00000120889 | TNFRSF10B | 1.426599 |
| ENSG00000150540 | HNMT | 1.95592 |
| ENSG00000152601 | MBNL1 | 1.176469 |
| ENSG00000154760 | SLFN13 | −1.28157 |
| ENSG00000172530 | BANP | 1.309606 |
| ENSG00000156535 | CD109 | 1.559254 |
| ENSG00000157782 | CABP1 | 1.713711 |
| ENSG00000160213 | CSTB | 1.278342 |
| ENSG00000166401 | SERPINB8 | −1.43123 |
| ENSG00000164086 | DUSP7 | 1.638783 |
| ENSG00000164124 | TMEM144 | 1.467621 |
| ENSG00000164211 | STARD4 | 1.209987 |
| ENSG00000164603 | C7orf60 | 1.357393 |
| ENSG00000165271 | NOL6 | −1.24346 |
| ENSG00000122376 | FAM35A | −1.14156 |
| ENSG00000102893 | PHKB | −1.14641 |
| ENSG00000166347 | CYB5A | 1.45897 |
| ENSG00000205544 | TMEM256 | 1.189937 |
| ENSG00000169129 | AFAP1L2 | 1.947398 |
| ENSG00000171723 | GPHN | 1.283108 |
| ENSG00000250565 | ATP6V1E2 | −1.20889 |
| ENSG00000169245 | CXCL10 | 9.610452 |
| ENSG00000170027 | YWHAG | 1.159436 |
| ENSG00000173548 | SNX33 | 2.356545 |
| ENSG00000066136 | NFYC | 1.152745 |
| ENSG00000118518 | RNF146 | 1.287798 |
| ENSG00000015171 | ZMYND11 | −1.29243 |
| ENSG00000138777 | PPA2 | 1.145937 |
| ENSG00000173801 | JUP | 2.053728 |
| ENSG00000167851 | CD300A | 1.262694 |
| ENSG00000120029 | C10orf76 | 1.265967 |
| ENSG00000059769 | DNAJC25 | −1.40426 |
| ENSG00000180353 | HCLS1 | −1.50923 |
| ENSG00000173826 | KCNH6 | −1.469 |
| ENSG00000023892 | DEF6 | −1.26219 |
| ENSG00000163687 | DNASE1L3 | 2.04322 |
| ENSG00000076382 | SPAG5 | −1.207 |
| ENSG00000176381 | PRR18 | 1.678804 |
| ENSG00000007237 | GAS7 | 1.534132 |
| ENSG00000177453 | NIM1K | 2.122782 |
| ENSG00000166949 | SMAD3 | −1.27483 |
| ENSG00000214517 | PPME1 | 1.243731 |
| ENSG00000184867 | ARMCX2 | 1.889032 |
| ENSG00000184371 | CSF1 | 1.683956 |
| ENSG00000185010 | F8 | 1.39024 |
| ENSG00000183621 | ZNF438 | 1.626425 |
| ENSG00000087086 | FTL | 1.396097 |
| ENSG00000184445 | KNTC1 | −1.38104 |
| ENSG00000166352 | C11orf74 | 1.82865 |
| ENSG00000146278 | PNRC1 | 1.218943 |
| ENSG00000188372 | ZP3 | 2.353015 |
| ENSG00000178498 | DTX3 | 1.251045 |
| ENSG00000169813 | HNRNPF | 1.21173 |
| ENSG00000176986 | SEC24C | 1.20218 |
| ENSG00000075651 | PLD1 | 3.178035 |
| ENSG00000152779 | SLC16A12 | −1.7143 |
| ENSG00000196131 | VN1R2 | 1.670713 |
| ENSG00000204248 | COL11A2 | 2.572336 |
| ENSG00000132676 | DAP3 | 1.077408 |
| ENSG00000132128 | LRRC41 | 1.271861 |
| ENSG00000126561 | STAT5A | 1.360053 |
| ENSG00000177105 | RHOG | 1.434685 |
| ENSG00000100243 | CYB5R3 | 1.38846 |
| ENSG00000116560 | SFPQ | −1.29434 |
| ENSG00000198113 | TOR4A | 1.208555 |
| ENSG00000088888 | MAVS | −1.13425 |
| ENSG00000100101 | NOL12 | −1.18394 |
| ENSG00000122786 | CALD1 | 1.73611 |
| ENSG00000185619 | PCGF3 | 1.277721 |
| ENSG00000135750 | KCNK1 | 2.225011 |
| ENSG00000117153 | KLHL12 | 1.164612 |
| ENSG00000148773 | MKI67 | −1.2805 |
| ENSG00000116815 | CD58 | 1.301381 |
| ENSG00000116171 | SCP2 | 1.133468 |
| ENSG00000158470 | B4GALT5 | 1.448768 |
| ENSG00000162368 | CMPK1 | 1.250972 |
| ENSG00000072401 | UBE2D1 | 1.255501 |
| ENSG00000106780 | MEGF9 | −1.53543 |
| ENSG00000204304 | PBX2 | −1.33416 |
| ENSG00000117115 | PADI2 | 2.229206 |
| ENSG00000065526 | SPEN | −1.16828 |
| ENSG00000165997 | ARL5B | 1.31121 |
| ENSG00000125821 | DTD1 | 1.164709 |
| ENSG00000133119 | RFC3 | −1.3687 |
| ENSG00000139618 | BRCA2 | −1.2248 |
| ENSG00000206527 | PTPLB | −1.22443 |
| ENSG00000143669 | LYST | 1.328396 |
| ENSG00000144597 | EAF1 | 1.162465 |
| ENSG00000214309 | MBLAC1 | −1.33186 |
| ENSG00000226025 | LGALS17A | −2.08926 |
| ENSG00000169136 | ATF5 | 2.107311 |
| ENSG00000032389 | TSSC1 | 1.094661 |

### REFERENCES

1. A. J. Levine, Spontaneous and inherited TP53 genetic alterations. Oncogene 40, 5975-5983 (2021).
2. A. J. Levine, p53: 800 million years of evolution and 40 years of discovery. Nat Rev Cancer 20, 471-480 (2020).
3. T. Guha, D. Malkin, Inherited TP53 Mutations and the Li-Fraumeni Syndrome. Cold Spring Harb Perspect Med 7 (2017).
4. S. W. Lowe, H. E. Ruley, T. Jacks, D. E. Housman, p53-dependent apoptosis modulates the cytotoxicity of anticancer agents. Cell 74, 957-967 (1993).
5. S. W. Lowe et al., p53 status and the efficacy of cancer therapy in vivo. Science 266, 807-810 (1994).
6. W. A. Freed-Pastor et al., Mutant p53 disrupts mammary tissue architecture via the mevalonate pathway. Cell 148, 244-258 (2012).
7. P. A. Muller et al., Mutant p53 drives invasion by promoting integrin recycling. Cell 139, 1327-1341 (2009).
8. P. A. Muller et al., Mutant p53 enhances MET trafficking and signalling to drive cell scattering and invasion. Oncogene 32, 1252-1265 (2013).
9. T. Cooks et al., Mutant p53 prolongs NF-kappaB activation and promotes chronic inflammation and inflammation-associated colorectal cancer. Cancer Cell 23, 634-646 (2013).
10. S. Boettcher et al., A dominant-negative effect drives selection of TP53 missense mutations in myeloid malignancies. Science 365, 599-604 (2019).
11. G. A. Lang et al., Gain of function of a p53 hot spot mutation in a mouse model of Li-Fraumeni syndrome. Cell 119, 861-872 (2004).
12. K. P. Olive et al., Mutant p53 gain of function in two mouse models of Li-Fraumeni syndrome. Cell 119, 847-860 (2004).
13. J. Bargonetti, C. Prives, Gain-of-function mutant p53: history and speculation. J Mol Cell Biol 11, 605-609 (2019).
14. P. A. Muller, K. H. Vousden, Mutant p53 in cancer: new functions and therapeutic opportunities. Cancer Cell 25, 304-317 (2014).
15. M. Oren, V. Rotter, Mutant p53 gain-of-function in cancer. Cold Spring Harb Perspect Biol 2, a001107 (2010).
16. M. Jennis et al., An African-specific polymorphism in the TP53 gene impairs p53 tumor suppressor function in a mouse model. Genes Dev 30, 918-930 (2016).
17. M. E. Murphy et al., A functionally significant SNP in TP53 and breast cancer risk in African-American women. NPJ Breast Cancer 3, 5 (2017).
18. J. I. Leu, M. E. Murphy, D. L. George, Mechanistic basis for impaired ferroptosis in cells expressing the African-centric S47 variant of p53. Proc Natl Acad Sci USA 116, 8390-8396 (2019).
19. J. I. Leu, M. E. Murphy, D. L. George, Functional interplay among thiol-based redox signaling, metabolism, and ferroptosis unveiled by a genetic variant of TP53. Proc Natl Acad Sci USA 117, 26804-26811 (2020).
20. K. S. Singh et al., African-centric TP53 variant increases iron accumulation and bacterial pathogenesis but improves response to malaria toxin. Nat Commun 11, 473 (2020).
21. K. Gnanapradeepan et al., Increased mTOR activity and metabolic efficiency in mouse and human cells containing the African-centric tumor-predisposing p53 variant Pro47Ser. Elife 9 (2020).
22. S. Kato et al., Understanding the function-structure and function-mutation relationships of p53 tumor suppressor protein by high-resolution missense mutation analysis. Proc Natl Acad Sci USA 100, 8424-8429 (2003).
23. J. Powers et al., A Rare TP53 Mutation Predominant in Ashkenazi Jews Confers Risk of Multiple Cancers. Cancer Res 80, 3732-3744 (2020).
24. K. Szoltysek et al., RRAD, IL4I, CDKN1A, and SERPINE1 genes are potentially co-regulated by NF-kappaB and p53 transcription factors in cells exposed to high doses of ionizing radiation. BMC Genomics 19, 813 (2018).
25. J. V. Gannon, R. Greaves, R. Iggo, D. P. Lane, Activating mutations in p53 produce a common conformational effect. A monoclonal antibody specific for the mutant form. EMBO J 9, 1595-1602 (1990).
26. Y. M. Zhu, D. Bradbury, N. Russell, Expression of different conformations of p53 in the blast cells of acute myeloblastic leukaemia is related to in vitro growth characteristics. Br J Cancer 68, 851-855 (1993).
27. P. W. Hinds, C. A. Finlay, A. B. Frey, A. J. Levine, Immunological evidence for the association of p53 with a heat shock protein, hsc70, in p53-plus-ras-transformed cell lines. Mol Cell Biol 7, 2863-2869 (1987).
28. O. Pinhasi-Kimhi, D. Michalovitz, A. Ben-Zeev, M. Oren, Specific interaction between the p53 cellular tumour antigen and major heat shock proteins. Nature 320, 182-184 (1986).
29. G. Di Minin et al., Mutant p53 reprograms TNF signaling in cancer cells through interaction with the tumor suppressor DAB2IP. Mol Cell 56, 617-629 (2014).
30. L. Weisz et al., Mutant p53 enhances nuclear factor kappaB activation by tumor necrosis factor alpha in cancer cells. Cancer Res 67, 2396-2401 (2007).
31. S. Chen et al., Arsenic Trioxide Rescues Structural p53 Mutations through a Cryptic Allosteric Site. Cancer Cell 39, 225-239 e228 (2021).
32. A. O. Giacomelli et al., Mutational processes shape the landscape of TP53 mutations in human cancer. Nat Genet 50, 1381-1387 (2018).
33. E. Kotler et al., A Systematic p53 Mutation Library Links Differential Functional Impact to Cancer Mutation Pattern and Evolutionary Conservation. Mol Cell 71, 178-190 e178 (2018).
34. B. Klimovich et al., p53 partial loss-of-function mutations sensitize to chemotherapy. Oncogene 41, 1011-1023 (2022).
35. F. Doffe et al., Identification and functional characterization of new missense SNPs in the coding region of the TP53 gene. Cell Death Differ 28, 1477-1492 (2021).
36. S. Raad et al., Blood functional assay for rapid clinical interpretation of germline TP53 variants. J Med Genet 58, 796-805 (2021).
37. Y. Zerdoumi et al., Germline TP53 mutations result into a constitutive defect of p53 DNA binding and transcriptional response to DNA damage. Hum Mol Genet 26, 2591-2602 (2017).
38. G. L. Bond et al., A single nucleotide polymorphism in the MDM2 promoter attenuates the p53 tumor suppressor pathway and accelerates tumor formation in humans. Cell 119, 591-602 (2004).
39. D. Walerych et al., Wild-type p53 oligomerizes more efficiently than p53 hot-spot mutants and overcomes mutant p53 gain-of-function via a “dominant-positive” mechanism. Oncotarget 9, 32063-32080 (2018).
39. National Research Council, Guide for the Care and Use of Laboratory Animals, (National Academies Press, Washington, DC, ed. 8, 2011).

All publications cited in this specification are incorporated herein by reference. While the invention has been described with reference to particular embodiments, it will be appreciated that modifications can be made without departing from the spirit of the invention. Such modifications are intended to fall within the scope of the appended claims.

1. A method of treating cancer in a subject in need thereof, the method comprising:
a) obtaining a tumor sample from the subject;
b) detecting expression levels of at least two genes in the tumor sample, wherein the genes are selected from:
CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYB5A, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A,
wherein a difference in the expression level of the genes as compared to a control level indicates the presence of a p53 hypomorphic variant; and
c) administering a treatment for the cancer to the subject.

2. (canceled)

3. A method of reducing, inhibiting, and/or preventing tumor metastasis in a subject in need thereof, the method comprising:
having obtained information about expression levels of two or more genes in a sample obtained from the subject, wherein the genes comprise
CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYB5A, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A,
wherein a difference in the expression level of the genes as compared to a control level indicates the presence of a p53 hypomorphic variant; and
c) administering a treatment for the cancer to the subject.

4. (canceled)

5. The method of claim 1, wherein the difference in the expression level of one or more of the following genes is an upregulation of one or more of:
CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYBSA, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, and DAP3.

6. The method of claim 1, wherein the difference in the expression level of one or more of the following genes is a downregulation of one or more of:
POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A.

7. The method of any one of claim 1, wherein the collection of genes consists of
CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYB5A, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A.

8. The method of claim 1, wherein the expression level of one or more of the genes is determined by detecting an RNA level, optionally by quantitative reverse transcription PCR (RT-qPCR) or RNA-Seq.

9. The method of claim 1, wherein the expression level of one or more of the genes is determined detecting a gene product/protein level, optionally by immunohistochemistry.

10. The method of claim 1, wherein the control is a normal tissue sample, or a level derived therefrom.

11. The method of claim 1, wherein the cancer is head and neck, esophageal, glioblastoma (GBM), kidney (clear cell and papillary cell carcinoma), colorectal, glioma, pancreatic ductal adenocarcinoma (PDAC), uveal melanoma, adrenocortical carcinoma, breast, neuroendocrine system, carcinoid, cervical, liver, lung, lymphoma, melanoma, ovarian, pancreatic, prostate, skin, stomach, testis, thyroid, or urothelial.

12. The method of claim 1, further comprising identification of a mutant TP53 allele in the sample.

13. The method of claim 12, wherein the mutant TP53 allele harbors a missense mutation.

14. The method of claim 1, wherein the encoded p53 has one or more of P47S (Pro47Ser, rs1800371), Y107H (Tyr107His, rs368771578), or G334R (Gly334Arg, rs78378222).

15. The method of claim 1, further comprising detecting an upregulation in NF-κB expression.

16. The method of claim 1, wherein the treatment comprises administering a chemotherapy, radiation therapy, immunotherapy, hormone therapy, stem cell or bone marrow transplant, and/or surgical resection.

17. The method of claim 1, wherein the sample is a tumor, whole blood, or peripheral blood mononuclear cells.

18. A composition comprising a collection of probes, primers, and/or antibodies suitable for detection of the expression and/or expression levels of a collection of genes, or their products, in a tumor sample, wherein the collection of genes comprises CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYB5A, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3m, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A.

19. The composition of claim 18, wherein the collection of genes consists of
CXCL10, FYN, ILDR2, PLD1, RAB38, COL11A2, NCALD, FFAR2, ANK1, SNX33, ZP3, VCAN, NPL, PADI2, KCNK1, NIM1K, ATF5, JUP, DNASE1L3, HNMT, AFAP1L2, ARMCX2, GDF15, C11orf74, CALD1, SRGN, CABP1, CSF1, PRR18, VN1R2, PPFIBP1, DUSP7, ZNF438, FKBP9, CD109, CPOX, GAS7, CSRNP1, TMEM144, CYB5A, TNFSF9, B4GALT5, RHOG, TNFRSF10B, SORD, FTL, TIMP1, F8, CYB5R3, SOX4, FAM127A, STAT5A, C7orf60, LYST, ARL5B, BANP, ELK1, CHIC2, CTSC, CD58, MXD1, RNF146, PAIP2, GPHN, CSTB, PCGF3, REPS1, LRRC41, C10orf76, CD300A, UBE2D1, DTX3, CMPK1, PPME1, PNRC1, ETFA, HNRNPF, TICAM1, STARD4, TOR4A, RAB7A, SEC24C, TMEM256, UBE2B, BST2, MBNL1, DTD1, KLHL12, EAF1, YWHAG, CHMP2B, NFYC, PPA2, ACO2, SCP2, PUM1, SNRPD3, ACAT1, TSSC1, DAP3m, POLDIP3, LSG1, MAVS, FAM35A, PHKB, CLEC4A, GPATCH1, SPEN, NOL12, NAA50, TNFAIP8, SPAG5, ATP6V1E2, PTPLB, BRCA2, NOL6, FUS, NUP85, DEF6, CNTRL, SMAD3, MKI67, SLFN13, DLGAP5, ZMYND11, SFPQ, HEATR6, MBLAC1, PBX2, RAD51, RFC3, KNTC1, DNAJC25, PDCL3, SERPINB8, KCNH6, HCLS1, MEGF9, BIN2, FAM65B, SLC16A12, TAPBPL, and LGALS17A.

20. The composition of claim 18, wherein at least one of the probes, primers, and/or antibodies is bound to a substrate.

21. The composition of claim 18, wherein at least one of the probes, primers, and/or antibodies is attached to a detectable label.

22. A kit comprising the composition of claim 18 and an apparatus for sample collection.

23-24. (canceled)
